SEQUENCE LISTING (79 sequences)

SEQ ID NO: 1 - protein, 8 aa, synthetic construct
GIDLSTNP

SEQ ID NO: 2 - protein, 8 aa, synthetic construct
IFADTGEI

SEQ ID NO: 3 - protein, 10 aa, synthetic construct
VRGSPSSANI

SEQ ID NO: 4 - protein, 8 aa, synthetic construct
QSVYNNNW

SEQ ID NO: 5 - skipped

SEQ ID NO: 6 - protein, 11 aa, synthetic construct
AGTYRRNSDNG

SEQ ID NO: 7 - protein, 8 aa, synthetic construct
GFSLSSYF

SEQ ID NO: 8 - protein, 7 aa, synthetic construct
HSDTGST

SEQ ID NO: 9 - protein, 14 aa, synthetic construct
ARDGGNDAYYYFNI

SEQ ID NO: 10 - protein, 6 aa, synthetic construct
QSISTA

SEQ ID NO: 11 - skipped

SEQ ID NO: 12 - protein, 13 aa, synthetic construct
QQGASARNIASNV

SEQ ID NO: 13 - protein, 8 aa, synthetic construct
GIDLSTNP

SEQ ID NO: 14 - protein, 8 aa, synthetic construct
IFADTGEI

SEQ ID NO: 15 - protein, 10 aa, synthetic construct
VRGSPSSANI

SEQ ID NO: 16 - protein, 8 aa, synthetic construct
QSVYNNNW

SEQ ID NO: 17 - skipped

SEQ ID NO: 18 - protein, 11 aa, synthetic construct
AGTYRRNSDNG

SEQ ID NO: 19 - protein, 8 aa, synthetic construct
GIDLSSGW

SEQ ID NO: 20 - protein, 7 aa, synthetic construct
INSGGSP

SEQ ID NO: 21 - protein, 5 aa, synthetic construct
GSRDF

SEQ ID NO: 22 - protein, 8 aa, synthetic construct
QSVYNNNY

SEQ ID NO: 23 - skipped

SEQ ID NO: 24 - protein, 13 aa, synthetic construct
SGGYDCDSADCSV

SEQ ID NO: 25 - protein, 8 aa, synthetic construct
GIDLSSGW

SEQ ID NO: 26 - protein, 7 aa, synthetic construct
INSYGRT

SEQ ID NO: 27 - protein, 5 aa, synthetic construct
GSRDF

SEQ ID NO: 28 - protein, 8 aa, synthetic construct
QSVYKNNY

SEQ ID NO: 29 - skipped

SEQ ID NO: 30 - protein, 13 aa, synthetic construct
GGGYDCDSADCSV

SEQ ID NO: 31 - protein, 115 aa, synthetic construct
QSVEESGGRLVTPGTPLTLTCTVSGIDLSTNPMGWVRQAPGKGLEWIGIIFADTGEIYYATWARGRFTISETSSTTVDLKITSPTAEDTATYFCVRGSPSSANIWGPGTLVTVSS

SEQ ID NO: 32 - protein, 111 aa, synthetic construct
AAVLTQTPSPVSAAVGGTVTISCQSSQSVYNNNWLGWYQQKPGQPPKLLIYRASTLASGVSSRFKGSGSGTQFTLTISGLECDDAATYYCAGTYRRNSDNGFGGGTEVVVK

SEQ ID NO: 33 - protein, 117 aa, synthetic construct
QSVEESGGRLVTPGGSLTLTCTVSGFSLSSYFMIWVRQAPGKGLEYIGIHSDTGSTDYASWAKGRFTISKTSTTVDLKMTSLTASDTATYFCARDGGNDAYYYFNIWGPGTLVTVSS

SEQ ID NO: 34 - protein, 111 aa, synthetic construct
AYDMTQTPASVEVAVGGTVTINCQASQSISTALAWYQQKPGQPPKPLIYEASKLASGVPSRFKGSGSGTEFILTISDLECADAATYYCQQGASARNIASNVFGGGTEVVVK

SEQ ID NO: 35 - protein, 115 aa, synthetic construct
QSVEESGGRLVTPGTPLTLTCTVSGIDLSTNPMGWVRQAPGKGLEWIGIIFADTGEIYYATWAKGRFTISETSSTTVDLKITSPTAEDTATYFCVRGSPSSANIWGPGTLVTVSS

SEQ ID NO: 36 - protein, 111 aa, synthetic construct
AAVLTQTPSPVSAAVGGTVTISCQSSQSVYNNNWLGWYQQKPGQPPKLLIYRASTLASGVPSRFKGSGSGTQFTFTISDLECDDAATYYCAGTYRRNSDNGFGGGTEVVVK

SEQ ID NO: 37 - protein, 108 aa, synthetic construct
QSVEESGGRLVTPGTPLTLTCTVSGIDLSSGWMNWVRQAPGGGLEWIGTINSGGSPYYARWAKGRFTLSTTSTTVDLKITSPTTEDTATYFCGSRDFWGPGTLVTVSS

SEQ ID NO: 38 - protein, 113 aa, synthetic construct
AQVLTQTASPVSAAVGGTVTINCQASQSVYNNNYLAWYQQKPGQPPKRLIYAASNLASGVSSRFKGSGSGTQFTLTISDLQCDDAATYYCSGGYDCDSADCSVFGGGTEVVVK

SEQ ID NO: 39 - protein, 108 aa, synthetic construct
QSVEESGGRLVTPGTPLTLTCTVSGIDLSSGWMNWVRQAPGKGLEWIGVINSYGRTYYARWAKGRFTISITSTTVDLKITSPTTEDTATYFCGSRDFWGPGTLVTVSS

SEQ ID NO: 40 - protein, 113 aa, synthetic construct
AQVLTQTASPVSAAVGSTVTINCQASQSVYKNNYLAWYQQKPGQPPKRLIYSASTLASGVSSRFKGSGSGTQFTLTISDVQCDDAATYYCGGGYDCDSADCSVFGGGTEVVVK

SEQ ID NO: 41 - protein, 457 aa, synthetic construct
METGLRWLLLVAVLKGVQCQSVEESGGRLVTPGTPLTLTCTVSGIDLSTNPMGWVRQAPGKGLEWIGIIFADTGEIYYATWARGRFTISETSSTTVDLKITSPTAEDTATYFCVRGSPSSANIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 42 - protein, 237 aa, synthetic construct
MDTRAPTQLLGLLLLWLPGATFAAVLTQTPSPVSAAVGGTVTISCQSSQSVYNNNWLGWYQQKPGQPPKLLIYRASTLASGVSSRFKGSGSGTQFTLTISGLECDDAATYYCAGTYRRNSDNGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 43 - protein, 459 aa, synthetic construct
METGLRWLLLVAVLKGVQCQSVEESGGRLVTPGGSLTLTCTVSGFSLSSYFMIWVRQAPGKGLEYIGIHSDTGSTDYASWAKGRFTISKTSTTVDLKMTSLTASDTATYFCARDGGNDAYYYFNIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 44 - protein, 237 aa, synthetic construct
MDTRAPTQLLGLLLLWLPGARCAYDMTQTPASVEVAVGGTVTINCQASQSISTALAWYQQKPGQPPKPLIYEASKLASGVPSRFKGSGSGTEFILTISDLECADAATYYCQQGASARNIASNVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 45 - protein, 457 aa, synthetic construct
METGLRWLLLVAVLKGVQCQSVEESGGRLVTPGTPLTLTCTVSGIDLSTNPMGWVRQAPGKGLEWIGIIFADTGEIYYATWAKGRFTISETSSTTVDLKITSPTAEDTATYFCVRGSPSSANIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 46 - protein, 237 aa, synthetic construct
MDTRAPTQLLGLLLLWLPGATFAAVLTQTPSPVSAAVGGTVTISCQSSQSVYNNNWLGWYQQKPGQPPKLLIYRASTLASGVPSRFKGSGSGTQFTFTISDLECDDAATYYCAGTYRRNSDNGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 47 - protein, 450 aa, synthetic construct
METGLRWLLLVAVLKGVQCQSVEESGGRLVTPGTPLTLTCTVSGIDLSSGWMNWVRQAPGGGLEWIGTINSGGSPYYARWAKGRFTLSTTSTTVDLKITSPTTEDTATYFCGSRDFWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 48 - protein, 239 aa, synthetic construct
MDTRAPTQLLGLLLLWLPGATFAQVLTQTASPVSAAVGGTVTINCQASQSVYNNNYLAWYQQKPGQPPKRLIYAASNLASGVSSRFKGSGSGTQFTLTISDLQCDDAATYYCSGGYDCDSADCSVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 49 - protein, 450 aa, synthetic construct
METGLRWLLLVAVLKGVQCQSVEESGGRLVTPGTPLTLTCTVSGIDLSSGWMNWVRQAPGKGLEWIGVINSYGRTYYARWAKGRFTISITSTTVDLKITSPTTEDTATYFCGSRDFWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 50 - protein, 239 aa, synthetic construct
MDTRAPTQLLGLLLLWLPGATFAQVLTQTASPVSAAVGSTVTINCQASQSVYKNNYLAWYQQKPGQPPKRLIYSASTLASGVSSRFKGSGSGTQFTLTISDVQCDDAATYYCGGGYDCDSADCSVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 51 - DNA, 1374 bp, synthetic construct
  source 1..1374: mol_type = other DNA
atggagactgggctgcgctggcttctcctcgtcgctgtgctcaaaggtgtccagtgtcagtcggtggaggagtccgggggtcgcctggtcacgcctgggacacccctgacactcacctgcacagtctctggaatcgacctcagtaccaatccaatgggctgggtccgccaggctccagggaaggggctggagtggatcggaatcatctttgctgacacgggtgaaatatactacgcgacctgggcgagaggccgattcaccatctccgaaacctcgtcgaccacggtggatctgaaaatcaccagtccgacagccgaggacacggccacctatttctgtgtcagagggtctccttctagtgctaacatctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 52 - DNA, 714 bp, synthetic construct
  source 1..714: mol_type = other DNA
atggacacgagggcccccactcagctgctggggctcctgctgctctggctcccaggtgccacatttgccgccgtgctgacccagactccatctcccgtgtctgcagctgtgggaggcacagtcaccatcagttgccagtccagtcagagtgtttataataacaactggttaggctggtatcagcagaaaccagggcagcctcccaaactcctgatctacagggcatccactctggcatctggggtctcatcgcggttcaaaggcagtggatctgggacacagttcactctcaccatcagcggccttgagtgtgacgatgctgcgacttactactgtgcaggcacttacaggcggaatagtgataatggtttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 53 - DNA, 1380 bp, synthetic construct
  source 1..1380: mol_type = other DNA
atggagactgggctgcgctggcttctcctggtcgctgtgctcaaaggtgtccagtgtcagtcggtggaggagtccgggggtcgcctggtcacgcctggaggatccctgacactcacctgcacagtctctggattctccctcagtagttacttcatgatctgggtccgccaggctccagggaaggggctggaatacatcggaatccatagtgatactggtagcacagactacgcgagctgggcgaaaggccgattcaccatctccaaaacctcgaccacggtggatctgaaaatgaccagtctgacagcttcagacacggccacctatttctgtgccagagatggtggtaatgatgcttattactactttaacatctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 54 - DNA, 714 bp, synthetic construct
  source 1..714: mol_type = other DNA
atggacacgagggcccccactcagctgctggggctcctgctgctctggctcccaggtgccagatgtgcctatgatatgacccagactccagcctctgtggaggtagctgtgggaggcacagtcaccatcaattgccaggccagccagagcattagcactgcattagcctggtatcagcagaaaccagggcagcctcccaagcccctgatctacgaagcatccaagctggcatctggggtcccatcgcggttcaaaggcagtggatctgggacagagttcattctcaccatcagcgacctggagtgtgccgatgctgccacttactactgtcaacagggtgctagtgctcgaaatattgcctcaaatgttttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 55 - DNA, 1374 bp, synthetic construct
  source 1..1374: mol_type = other DNA
atggagactgggctgcgctggcttctcctcgtcgctgtgctcaaaggtgtccagtgtcagtcggtggaggagtccgggggtcgcctggtcacgcctgggacacccctgacactcacctgcacagtctctggaatcgacctcagtaccaatccaatgggctgggtccgccaggctccagggaaggggctggaatggatcggaatcatctttgctgacacgggtgaaatatactacgcgacctgggcgaaaggccgattcaccatctccgaaacctcgtcgaccacggtggatctgaaaatcaccagtccgacagccgaggacacggccacctatttctgtgtcagagggtctccttctagtgctaacatctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 56 - DNA, 714 bp, synthetic construct
  source 1..714: mol_type = other DNA
atggacacgagggcccccactcagctgctggggctcctgctgctctggctcccaggtgccacatttgccgccgtgctgacccagactccatctcccgtgtctgcagctgtgggaggcacagtcaccatcagttgccagtccagtcagagtgtttataataacaactggttaggctggtatcagcagaaaccagggcagcctcccaaactcctgatctacagggcatccactctggcatctggggtcccatcgcggttcaaaggcagtggatctgggacacagttcactttcaccatcagcgaccttgagtgtgacgatgctgcgacttactactgtgcaggcacttacaggcggaatagtgataatggtttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 57 - DNA, 1353 bp, synthetic construct
  source 1..1353: mol_type = other DNA
atggagactgggctgcgctggcttctcctggtcgctgtgctcaaaggtgtccagtgtcagtcggtggaggagtccgggggtcgcctggtcacgcctgggacacccctgacactcacctgcacagtttctggaatcgacctcagtagcggctggatgaattgggtccgccaggctccagggggggggctggaatggatcggaaccattaatagtggtggtagcccatactacgcgcgctgggcgaaaggccgattcaccctctccacaacctcgaccacggtggatctgaagatcaccagtccgacaaccgaagacacggccacctatttctgtggcagccgggacttctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 58 - DNA, 720 bp, synthetic construct
  source 1..720: mol_type = other DNA
atggacacgagggcccccactcagctgctggggctcctgctgctctggctcccaggtgccacatttgcccaagtgctgacccagactgcatcgcccgtgtctgcagctgtgggaggcacagtcaccatcaattgccaggccagtcagagtgtttataacaacaactatttagcctggtatcagcagaaaccagggcagcctcccaagcgcctgatctatgctgcatccaacctggcatctggggtctcatcgcggttcaaaggcagtggatctgggacacagttcactctcaccatcagcgacctacagtgtgacgatgctgccacttactactgttcaggcggttatgattgtgatagtgctgattgtagcgttttcggcggggggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 59 - DNA, 1353 bp, synthetic construct
  source 1..1353: mol_type = other DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagtagcggctggatgaactgggtccgccaggctccagggaagggactggagtggatcggagtcattaatagttatggtaggacatactacgcgcgctgggcgaaaggccgattcaccatctccattacctcgaccacggtggatctgaagatcaccagtccgacaaccgaagacacggccacctatttctgtggcagccgggacttctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 60 - DNA, 720 bp, synthetic construct
  source 1..720: mol_type = other DNA
atggacacgagggcccccactcagctgctggggctcctgctgctctggctcccaggtgccacatttgcccaagtgctgacccagactgcatcgcccgtgtctgcagctgtgggaagcacagtcaccatcaattgccaggccagtcagagtgtttataagaacaactacttagcctggtatcagcagaaaccagggcagcctcccaagcgcctgatctattctgcatccactctggcatctggggtctcatcgcggttcaaaggcagtggatctgggacacagttcactctcaccatcagcgacgtgcagtgtgacgatgctgccacttactactgtggaggcggttatgattgtgatagtgctgattgtagcgttttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 61 - protein, 93 aa, synthetic construct
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 62 - DNA, 279 bp, synthetic construct
  source 1..279: mol_type = other DNA
atgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagag

SEQ ID NO: 63 - protein, 114 aa, synthetic construct
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 64 - DNA, 342 bp, synthetic construct
  source 1..342: mol_type = other DNA
atgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccc

SEQ ID NO: 65 - protein, 93 aa, synthetic construct
MLTELEKALNSAADVYHKYSLIKGNFHAVYRDDLKKLLETECPQYIRKKGADVWFKELDINTDGAVNFQEFAILVIKMGVAAHKKSHEESHKE

SEQ ID NO: 66 - DNA, 279 bp, synthetic construct
  source 1..279: mol_type = other DNA
atgttgaccgagctggagaaagccttgaactctgccgccgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcgccattctggtgataaagatgggcgtggcagcccacaaaaaaagccatgaagaaagccacaaagag

SEQ ID NO: 67 - protein, 114 aa, synthetic construct
MTCKMSQLARNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 68 - DNA, 342 bp, synthetic construct
  source 1..342: mol_type = other DNA
atgacttgcaaaatgtcgcagctggcacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccc

SEQ ID NO: 69 - DNA, 19 bp, synthetic construct
  source 1..19: mol_type = other DNA
taatacgactcactatagg

SEQ ID NO: 70 - DNA, 25 bp, synthetic construct
  source 1..25: mol_type = other DNA
ggaattgtgagcggataacaattcc

SEQ ID NO: 71 - DNA, 23 bp, synthetic construct
  source 1..23: mol_type = other DNA
tttgtttaactttaagaaggaga

SEQ ID NO: 72 - DNA, 33 bp, synthetic construct
  source 1..33: mol_type = other DNA
atggctagcatgactggtggacagcaaatgggt

SEQ ID NO: 73 - DNA, 24 bp, synthetic construct
  source 1..24: mol_type = other DNA
gattacaaggacgacgatgacaag

SEQ ID NO: 74 - DNA, 18 bp, synthetic construct
  source 1..18: mol_type = other DNA
caccaccaccaccaccac

SEQ ID NO: 75 - DNA, 790 bp, synthetic construct
  source 1..790: mol_type = other DNA
atggattacaaggacgacgatgacaagttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtaataatacgactcactataggggaattgtgagcggataacaattcccctctagaaataattttgtttaactttaagaaggagatatacatatggctagcatgactggtggacagcaaatgggtatgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccccaccaccaccaccaccactaa

SEQ ID NO: 76 - DNA, 790 bp, synthetic construct
  source 1..790: mol_type = other DNA
atggattacaaggacgacgatgacaagttgaccgagctggagaaagccttgaactctgccgccgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcgccattctggtgataaagatgggcgtggcagcccacaaaaaaagccatgaagaaagccacaaagagtaataatacgactcactataggggaattgtgagcggataacaattcccctctagaaataattttgtttaactttaagaaggagatatacatatggctagcatgactggtggacagcaaatgggtatgacttgcaaaatgtcgcagctggcacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccccaccaccaccaccaccactaa

SEQ ID NO: 77 - DNA, 790 bp, synthetic construct
  source 1..790: mol_type = other DNA
atggattacaaggacgacgatgacaagttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtaataatacgactcactataggggaattgtgagcggataacaattcccctctagaaataattttgtttaactttaagaaggagatatacatatggctagcatgactggtggacagcaaatgggtatgacttgcaaaatgtcgcagctggcacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccccaccaccaccaccaccactaa

SEQ ID NO: 78 - DNA, 282 bp, synthetic construct
  source 1..282: mol_type = other DNA
atgctgaccgaactggaaaaagcgctgaacagcattattgatgtgtatcataaatatagcctgattaaaggcaactttcatgcggtgtatcgcgatgatctgaaaaaactgctggaaaccgaatgcccgcagtatattcgcaaaaaaggcgcggatgtgtggtttaaagaactggatattaacaccgatggcgcggtgaactttcaggaatttctgattctggtgattaaaatgggcgtggcggcgcataaaaaaagccatgaagaaagccataaagaataa

SEQ ID NO: 79 - DNA, 345 bp, synthetic construct
  source 1..345: mol_type = other DNA
atgacctgcaaaatgagccagctggaacgcaacattgaaaccattattaacacctttcatcagtatagcgtgaaactgggccatccggataccctgaaccagggcgaatttaaagaactggtgcgcaaagatctgcagaactttctgaaaaaagaaaacaaaaacgaaaaagtgattgaacatattatggaagatctggataccaacgcggataaacagctgagctttgaagaatttattatgctgatggcgcgcctgacctgggcgagccatgaaaaaatgcatgaaggcgatgaaggcccgggccatcatcataaaccgggcctgggcgaaggcaccccgtaa

(19) United States
(12) Patent Application Publication
(10) Pub. No.: US 2025/0093368 A1
(43) Pub. Date: Mar. 20, 2025

(54) ANTIBODIES AGAINST S100A8 AND S100A9, HETERODIMER OF S100A8 AND S100A9, DETECTION KIT AND USE OF THE ANTIBODIES, HETERODIMER AND DETECTION KIT

(71) Applicant: BEIJING INSTITUTE OF HEART LUNG AND BLOOD VESSEL DISEASES, Beijing (CN)

(72) Inventors: Yulin LI, Beijing (CN); Jie DU, Beijing (CN); Shijuan GAO, Beijing (CN)

(21) Appl. No.: 18/493,108

(22) Filed: Oct. 24, 2023

(30) Foreign Application Priority Data
Sep. 18, 2023 (CN) 202311203510.4

(51) Int. Cl. G01N 33/68; C07K 14/52; C07K 16/24

(52) U.S. Cl. CPC G01N 33/6863; C07K 14/52; C07K 16/24; G01N 2333/52; G01N 2800/24

(57) ABSTRACT

The present application provides an antibody or an antigen-binding fragment thereof, which can effectively bind to S100A8 or S100A9. The present application further provides an S100A8/S100A9 heterodimer, wherein by co-expressing mutant S100A9 with wild type S100A8, the generation of homodimers can be effectively reduced, and a soluble heterodimer with high purity can be obtained. The present application further provides an S100A8/S100A9 heterodimer detection kit, which comprises the antibody or the antigen-binding fragment thereof, and the S100A8/S100A9 heterodimer of the present application, and has the characteristics of high sensitivity, strong specificity and simple operation.

### CROSS-REFERENCE TO RELATED APPLICATIONS

This application claims priority to Chinese Patent Application No. 202311203510.4, filed with the China National Intellectual Property Administration on Sep. 18, 2023, the entire contents of which are hereby incorporated by reference in their entirety for all purpose.

### TECHNICAL FIELD

The present application relates to the field of molecular biology, in particular to an antibody or an antigen-binding fragment thereof that binds to S100A8 or S100A9, a heterodimer of S100A8 and S100A9, and a kit, and further in particular to the use of the antibody or the antigen-binding fragment thereof, the heterodimer and the kit.

### SEQUENCE LISTING

This application contains a Sequence Listing that has been submitted electronically as an ASCII text file named “57297-0002001_SequenceListing_2023_10_14.” The ASCII text file, created on Oct. 14, 2023, is 84 kilobytes in size. The material in the ASCII text file is hereby incorporated by reference in its entirety.

### BACKGROUND ART

S100 protein family is a class of calcium-binding proteins with low molecular weight that exist in the form of homodimers or heterodimers to maintain protein stability and can play a variety of biological functions through the regulation of Ca²⁺ and the interaction with target proteins. Among them, S100A8 consists of 93 amino acid residues and S100A9 consists of 113 amino acids.

Based on the distribution characteristics of hydrophobic groups on the surface of S100A8 and S100A9, they are more likely to form a heterodimer, and the stability of heterodimer is much greater than that of a homodimer. The heterodimer of S100A8 and S100A9 (hereafter referred to as S100A8/S100A9 heterodimer), after release from neutrophils, mainly exerts its effector function by binding to two pattern recognition receptors: Toll-like receptor 4 (TLR4) and receptor for advanced glycation endproducts (RAGE). As a dangerous signal of inflammatory reaction, S100A8/S100A9 are involved in the regulation of a variety of inflammatory and immune responses, and are considered to be inflammatory markers and potential therapeutic targets. A significant increase in S100A8/S100A9 was observed in a variety of disease states such as infectious and non-infectious inflammations, including sepsis, systemic lupus erythematosus, chronic obstructive pulmonary disease and tumors. Recently, our work published in Circulation, an authoritative journal in the cardiovascular field, and the follow-up work by several international teams have confirmed (Circulation, 2019, 140 (9): 751-764): S100A8/S100A9 increased rapidly in the early stage of myocardial ischemia/reperfusion injury, and the treatment with the neutralizing antibody against S100A9 could significantly slow down myocardial ischemia/reperfusion injury; and it has also been confirmed that S100A8/S100A9 had the important predictive value in early warning after interventional therapy for acute myocardial infarction.

However, there is currently no kit that can specifically detect only S100A8/S100A9 heterodimer. Therefore, the establishment of a novel kit to detect the level of S100A8/S100A9 heterodimer in a clinical sample, as a method for early warning of systemic inflammatory response, has important clinical and scientific research value.

Meanwhile, the development of antibodies against S100A8 and S100A9 requires high-purity proteins as antigens, and detection kits also require a high-purity S100A8/S100A9 heterodimeric protein as a standard. However, both S100A8 and S100A9 themselves are likely to form a homodimeric or even a high polymeric (such as tetrameric) protein product, which makes it difficult to form a heterodimer or greatly reduce the formation of heterodimers when expressed in vitro, making it difficult to obtain high-purity and high-quality heterodimers. Therefore, there is an urgent need to use means such as genetic engineering to express S100A8/S100A9 heterodimer with high purity in vitro to assist in the establishment of a high-specificity detection kit for S100A8/S100A9 heterodimer and also facilitate in-depth study on the structure and biological function of this dimer.

It should be noted that methods described in this section are not necessarily methods that have been previously conceived or employed. It should not be assumed that any of the methods described in this section is considered to be the prior art just because they are included in this section, unless otherwise indicated expressly. Similarly, the problem mentioned in this section should not be considered to be universally recognized in any prior art, unless otherwise indicated expressly.

### SUMMARY OF THE APPLICATION

In order to solve the above technical problems, the present application provides an antibody or an antigen-binding fragment thereof that binds to S100A8 or S100A9, wherein the antibody or the antigen-binding fragment at least comprises one group or more groups selected from groups (i)-(iii) and/or groups (iv)-(v): (i) sequences as shown in SEQ ID NOs: 1, 2, 3, 4 and 6, and RAS; (ii) sequences as shown in SEQ ID NOs: 7, 8, 9, 10 and 12, and EAS; (iii) sequences as shown in SEQ ID NOs: 13, 14, 15, 16 and 18, and RAS; (iv) sequences as shown in SEQ ID NOs: 19, 20, 21, 22 and 24, and AAS; and/or (v) sequences as shown in SEQ ID NOs: 25, 26, 27, 28 and 30, and SAS.

According to an embodiment of the present application, a polynucleotide is further provided, wherein the polynucleotide encodes the antibody or the antigen-binding fragment thereof of the present application.

According to an embodiment of the present application, a recombinant vector is further provided, wherein the recombinant vector comprises the polynucleotide of the present application.

According to an embodiment of the present application, a host cell is further provided, wherein the host cell comprises the polynucleotide of the present application, and/or the recombinant vector of the present application.

According to an embodiment of the present application, a polypeptide is further provided, wherein the polypeptide comprises an amino acid sequence having at least 80%, 85%, 90%, 95%, 98% or 99% identity to a sequence as shown in any one of SEQ ID NO: 61, SEQ ID NO: 65, SEQ ID NO: 63 or SEQ ID NO: 67. In some preferred embodiments, the polypeptide comprises a sequence as shown in any one of SEQ ID NO: 61, SEQ ID NO: 65, SEQ ID NO: 63 or SEQ ID NO: 67. By mutating hydrophilic amino acid E at position 9 of A chain of human S100A9 protein to hydrophobic amino acid A, the formation of the homodimer and high polymer of S100A9 is effectively reduced, which is beneficial to obtain high-purity S100A9 protein.

According to an embodiment of the present application, a heterodimer of S100A8 and S100A9 is further provided, wherein the heterodimer comprises amino acid sequences having at least 80%, 85%, 90%, 95%, 98% or 99% identity to sequences as shown in SEQ ID NO: 61 and SEQ ID NO: 67. In some preferred embodiments, the heterodimer comprises sequences as shown in SEQ ID NO: 61 and SEQ ID NO: 67. By mutating amino acid E at position 9 of A chain of human S100A9 protein to amino acid A, and co-expressing the mutant S100A9 with wild type S100A8, the generation of homodimers can be effectively reduced, and a soluble heterodimer with high purity can be obtained; furthermore, these mutations do not affect the activity of the protein, and the resulting recombinant protein has good activity.

The high-purity S100A8 protein, S100A9 protein and S100A8/S100A9 heterodimer provided by the present application lay a foundation for the study of the functions of these proteins, and the purified high-purity protein can be used as a standard in a detection kit or as an antigen in the development of a specific antibody, showing good application prospects.

According to an embodiment of the present application, a polynucleotide is further provided, wherein the polynucleotide encodes the polypeptide of the present application, or encodes the heterodimer of the present application.

According to an embodiment of the present application, an expression vector is further provided, wherein the expression vector comprises the polynucleotide of the present application.

According to an embodiment of the present application, an expression vector encoding the heterodimer of the present application is further provided, wherein the expression vector comprises an S100A8 fusion gene and an S100A9 fusion gene; the S100A8 fusion gene comprises a first promoter and S100A8 gene which are operably linked, and the S100A8 gene has a nucleic acid sequence as shown in SEQ ID NO: 62; and the S100A9 fusion gene comprises a second promoter and S100A9 gene which are operably linked, and the S100A9 gene has a nucleic acid sequence as shown in SEQ ID NO: 68. After the expression vector provided by the present application is introduced into a host cell, an active heterodimer can be formed in the host cell, and a large number of S100A8/S100A9 heterodimer with high activity and high purity can be obtained by purification, with the expression amount reaching 10 mg/L and the purity reaching 90% or more. In some embodiments, the first promoter and the second promoter are the same. By respectively driving the expression of S100A8 gene and S100A9 gene using two promoters in a vector, the co-expression of the S100A8 gene and the S100A9 gene can be realized. Moreover, promoters with the same or similar transcription capability can be selected to make the expression levels of the two genes balanced, thereby facilitating the promotion of the generation of the heterodimer.

According to an embodiment of the present application, a host cell is further provided, wherein the host cell comprises the polynucleotide of the present application, and/or the expression vector of the present application.

According to an embodiment of the present application, a kit is further provided, wherein the kit comprises the antibody or the antigen-binding fragment thereof of the present application, the polypeptide of the present application, and/or the heterodimer of the present application. In some embodiments, the kit comprises a capture antibody and a labeled antibody. According to the S100A8/S100A9 heterodimer detection kit provided by the present application, the standard in the kit is the S100A8/S100A9 heterodimer provided by the present application, and the antibody pairing strategy is used, that is, the capture antibody is an anti-S100A8 antibody and the labeled antibody is an anti-S100A9 antibody, or the capture antibody is an anti-S100A9 antibody and the labeled antibody is an anti-S100A8 antibody, and the kit has the characteristics of high sensitivity, strong specificity and simple operation.

According to an embodiment of the present application, the use of the antibody or the antigen-binding fragment thereof of the present application, and/or the polypeptide of the present application in the preparation of a reagent, a kit, an antibody chip or an antibody probe for detecting S100A8, S100A9, and a dimer of S100A8 and S100A9 is further provided.

According to an embodiment of the present application, the use of the heterodimer of the present application in the preparation of a reagent and a kit for detecting a dimer of S100A8 and S100A9 is further provided.

According to an embodiment of the present application, the use of the antibody or the antigen-binding fragment thereof of the present application, the polypeptide of the present application, and/or the heterodimer of the present application in the preparation of a reagent, a kit, an antibody chip or an antibody probe for detecting a disease or a non-disease symptom.

According to an embodiment of the present application, a method for detecting a disease or a non-disease symptom is further provided, wherein the method comprises the following steps: obtaining a sample of a subject; using the antibody or the antigen-binding fragment thereof of the present application, the polypeptide of the present application, the heterodimer of the present application, and/or the kit of the present application to detect the expression level of the dimer of S100A8 and S100A9 in the sample; and judging that the subject has the disease or the non-disease symptom according to the expression level of the dimer of S100A8 and S100A9 in the sample.

It should be understood that the content described in this section is not intended to identify critical or important features of the examples of the present application, and is not used to limit the scope of the present application. Other features of the present application will be easily understood through the following description.

### BRIEF DESCRIPTION OF THE DRAWINGS

The accompanying drawings exemplarily show examples and form a part of the specification, and are used to explain exemplary implementations of the examples together with a written description of the specification. The examples shown are merely for illustrative purposes and do not limit the scope of the claims. Throughout the accompanying drawings, the same reference numerals denote similar but not necessarily same elements.

FIG. 1 shows a schematic diagram of the interaction between the human S100A8s in the homodimer at mutation sites 112 and 113 in example 1.1.

FIG. 2 shows a schematic diagram of the interaction between the human S100A8s in the homodimer at mutation site L72 in example 1.1.

FIG. 3 shows a schematic diagram of the interaction between homodimers of human S100A9 at mutation site E9 in example 1.1.

FIG. 4 shows vector maps of plasmid 3 and plasmid 4 in example 1.1; wherein the maps sequentially represent plasmid 3 and plasmid 4 from top to bottom.

FIG. 5 shows SDS-PAGE electrophoretograms of the expressed and purified recombinant proteins from plasmid 1, plasmid 2, plasmid 3, plasmid 4, mutant plasmid 1 and mutant plasmid 2 in example 1.2; wherein panels A-F show plasmid 1, plasmid 2, mutant plasmid 1, mutant plasmid 2, plasmid 3 and plasmid 4, respectively.

FIG. 6 shows a vector map of plasmid 5 in example 1.3.

FIG. 7 shows an SDS-PAGE electrophoretogram of the expressed and purified recombinant protein from plasmid 5 in example 1.3.

FIG. 8 shows an SDS-PAGE electrophoretogram of the S100A8/S100A9 heterodimer protein standard in example 1.3, wherein lanes BSA, MK and RD represent bovine serum albumin, Marker and standard, respectively.

FIG. 9 shows a size exclusion chromatography-high performance liquid chromatogram of the S100A8/S100A9 heterodimer protein standard in example 1.3; wherein the upper panel shows the peak diagram of Maker K, and the lower panel shows the peak diagram of the S100A8/S100A9 protein.

FIG. 10 shows a size exclusion chromatography-high performance liquid chromatogram of the purified S100A8/S100A9-1 recombinant protein of the present application in example 1.3; wherein the upper panel shows the peak diagram of Maker K, and the lower panel shows the peak diagram of the S100A8/S100A9-1 recombinant protein.

FIG. 11 shows a curve graph of IL-6 secretion by A375 cells induced by the treatment with different concentrations of the S100A8/S100A9-1 recombinant protein in example 1.4.

FIG. 12 shows an SDS-PAGE electrophoretogram of the prepared S100A8 recombinant protein in example 2.1.

FIG. 13 shows an SDS-PAGE electrophoretogram of the prepared S100A9 recombinant protein in example 2.1.

FIG. 14 shows a diagram of ELISA detection results of the serum after seven immunizations of rabbits with S100A8 in example 2.2.

FIG. 15 shows pictures of Dot Blot detection results of the S100A8 antiserum in different concentrations of patient sera in example 2.2.

FIG. 16 shows a diagram of ELISA detection results of the serum after four immunizations of rabbits with S100A9 in example 2.2.

FIG. 17 shows pictures of Dot Blot detection results of the S100A9 antiserum in different concentrations of patient sera in example 2.2.

FIG. 18 shows a diagram of detection results of double-positive clones obtained by screening S100A8-positive B cell supernatant with the expressed $100A8 antigen and the commercial S100A8 protein in example 2.3 (OD>0.7).

FIG. 19 shows pictures of Dot blot detection results of the S100A8-positive B cell supernatant in patient sera in example 2.3.

FIG. 20 shows a diagram of detection results of double-positive clones obtained by screening S100A9-positive B cell supernatant with the expressed S100A9 antigen and the commercial S100A9 protein in example 2.3 (OD>0.7).

FIG. 21 shows pictures of Dot blot detection results of the S100A9-positive B cell supernatant in patient sera in example 2.3.

FIG. 22 shows a diagram of ELISA detection results of the LEM (linear expression module) supernatant of S100A8 using the S100A8 recombinant protein in example 2.3.

FIG. 23 shows a diagram of ELISA detection results of the LEM supernatant of S100A8 using the commercial S100A8 protein in example 2.3; wherein PC represents positive control and NC represents negative control.

FIG. 24 shows a diagram of ELISA detection results of the LEM supernatant of S100A8 using the S100A8/S100A9 heterodimer protein in example 2.3.

FIG. 25 shows diagrams of affinity sorting results of the LEM supernatant of S100A8 in example 2.3.

FIG. 26 shows a diagram of ELISA detection results of the LEM supernatant of S100A9 using the S100A9 recombinant protein in example 2.3.

FIG. 27 shows a diagram of ELISA detection results of the LEM supernatant of S100A9 using the commercial S100A9 protein in example 2.3; wherein PC represents positive control and NC represents negative control.

FIG. 28 shows a diagram of ELISA detection results of the LEM supernatant of S100A9 using the S100A8/S100A9 heterodimer protein in example 2.3.

FIG. 29 shows diagrams of affinity sorting results of the LEM supernatant of S100A9 in example 2.3.

FIG. 30 shows a diagram of ELISA detection results of 4 antibody pairs (5H10-7C4, 3D8-8A11, 3D8-7C4 and 5H10-8A11) in which the coating antibody is an anti-S100A8 antibody and the detecting antibody is an anti-S100A9 antibody under a low-concentration S100A8/S100A9 in example 3.

FIG. 31 shows a diagram of ELISA detection results of 2 antibody pairs (5H10-7C4 and 2C2-7C4) in which the coating antibody is an anti-S100A8 antibody and the detecting antibody is an anti-S100A9 antibody under a high-concentration S100A8/S100A9 in example 3.

FIG. 32 shows a diagram of ELISA detection results of 4 antibody pairs (7C4-5H10, 7C4-3D8, 8A11-3D8 and 8A11-5H10) in which the coating antibody is an anti-S100A9 antibody and the detecting antibody is an anti-S100A8 antibody under a low-concentration S100A8/S100A9 in example 3.

FIG. 33 shows a diagram of ELISA detection results of 2 antibody pairs (7C4-5H10 and 7C4-2C2) in which the coating antibody is an anti-S100A9 antibody and the detecting antibody is an anti-S100A8 antibody under a low-concentration S100A8/S100A9 in example 3.

FIG. 34 shows diagrams of standard curves obtained from the antibody pair 5H10-7C4 in 6 batches of experiments in example 4.4.

FIG. 35 shows a diagram of a relationship between the serum S100A8/S100A9 level detected using the antibody pair 5H10-7C4 and whether a subject has a disease (healthy individuals or patients with coronary heart disease) in example 4.4.

FIG. 36 shows a diagram of a relationship between the serum S100A8/S100A9 level detected using the antibody pair 5H10-7C4 and the serum S100A8/S100A9 level detected using the commercial S100A8/100A9 kit in example 4.4.

FIG. 37 shows a diagram of detection results of the binding ability of the antibody pair 5H10-7C4 to different S100A8/S100A9 heterodimers (the prepared S100A8/S100A9 and S100A8/S100A9-1 of the present application, and the commercial S100A8/S100A9) in example 5.2.

### DETAILED DESCRIPTION OF EMBODIMENTS

Unless otherwise indicated, all numbers representing content, concentration, ratio, mass, volume, time, temperature, thickness, technical effect, and so forth as used in the description and claims are to be understood as being modified in any case by the term “about” or “approximately”. Accordingly, unless indicated to the contrary, numerical parameters as set forth in the following specification and appended claims are approximations. For those of ordinary skill in the art, each numerical parameter may vary depending upon the desired properties and effects sought to be obtained by the present disclosure and should be construed in light of the number of significant figures and conventional rounding methods or in a manner understood by those of ordinary skill in the art.

Although the broad range of the numerical values and the parameters which are approximations of the present disclosure are as set forth herein, the numerical values as set forth in the specific examples are given as precisely as possible. However, any numerical value inherently contains certain errors, which are inevitably caused by the standard deviation found in their respective test measurements. Every numerical range given throughout the present description will include every narrower numerical range that falls within such a broader numerical range, as if such narrower numerical ranges are all expressly written herein.

Unless otherwise indicated or contradicts the context, the terms or expressions used herein should be read in conjunction with the entire content of the present disclosure and as understood by those of ordinary skill in the art. All technical and scientific terms used herein have the same meanings as commonly understood by those of ordinary skill in the art, unless otherwise defined.

When used herein, the expression “A and/or B” includes three cases: (1) A; (2) B; and (3) A and B. The expression “A, B and/or C” includes seven cases: (1) A; (2) B; (3) C; (4) A and B; (5) A and C; (6) B and C; and (7) A, B and C. The meaning of similar expressions can be deduced in the same way.

In the present application, the terms “nucleic acid” and “polynucleotide” are used interchangeably, and refer to polymerization forms of nucleotides of any length, including deoxyribonucleotides, ribonucleotides, combinations thereof, and analogs thereof.

In the present application, the terms “polypeptide” and “peptide” are used interchangeably, and refer to polymers of amino acids of any length. Therefore, polypeptides, oligopeptides, proteins, antibodies and enzymes are all included in the definition of polypeptide.

As described in the present application, the “fragment” of a sequence refers to a portion of a sequence. For example, the fragment of a nucleic acid sequence refers to a portion of the nucleic acid sequence, and the fragment of an amino acid sequence refers to a portion of the amino acid sequence.

In the present application, the terms such as “S100A8/S100A9”, “S100A8/S100A9 dimer”, “S100A8/S100A9 heterodimer”, “S100A8/S100A9 polypeptide”, “S100A8/S100A9 protein” or “the heterodimer of S100A8 and S100A9” have the same meaning, are used interchangeably, and refer to protein complexes in the form of a heterodimer formed by the hydrophobic interaction of calcium-binding protein A8 (S100A8) and calcium-binding protein A9 (S100A9) in the presence of calcium and zinc ions.

In the present application, the term “antibody” refers to a specific immunoglobulin targeting an antigenic site. The antibody in the present application refers to an antibody that specifically binds to S100A8 or S100A9, and can be manufactured according to well-known methods in the art. The forms of the antibody include polyclonal or monoclonal antibodies, antibody fragments (such as Fab, Fab′, F(ab′) 2 and Fv fragments), single chain Fv (scFv) antibodies, multispecific antibodies (such as bispecific antibodies), monospecific antibodies, monovalent antibodies, chimeric antibodies, humanized antibodies, human antibodies, fusion proteins comprising antigen-binding sites of antibodies, and any other modified immunoglobulin molecules comprising antigen-binding sites, as long as the antibodies exhibit the desired biological binding activity.

In the present application, the term “gene” refers to a complete nucleotide sequence required to produce a polypeptide chain or functional RNA. Therefore, the expression of a gene includes transcription and stable accumulation of a coding RNA (mRNA) or functional RNA derived from the gene, and also includes translation of the mRNA into a polypeptide or protein.

It should be noted that, in the context of the present application, the term “upstream” refers to the 5′ end of a gene or the N-terminus of a protein, the term “downstream” refers to the 3′ end of a gene or the C-terminus of a protein, and the expression “from downstream to downstream” refers to from the 5′ end to the 3′ end or from the N-terminus to the C-terminus.

In the present application, the terms “exogenous” and “heterologous” are used interchangeably, and refer to a source different an innate (original) organism, such as an organism derived from another species. In the present application, the expression “heterologous gene” or “exogenous gene” refers to a gene that is not naturally present in a host organism and that is introduced into the host organism by gene transfer, for example, the S100A8 gene and the S100A9 gene in the present application are not naturally present in Escherichia coli.

In the present application, the expression “fusion gene” refers to any gene that is not a natural gene, and comprises regulatory sequences and coding sequences that are not present together in a natural condition. Therefore, the fusion gene can comprise regulatory sequences and coding sequences derived from different organism sources, or can comprise regulatory sequences and coding sequences derived from the same organism source but arranged in a manner different from naturally occurring.

In the present application, the expression “fusion protein” is used in its conventional meaning, and refers to a protein consisting of a plurality of polypeptides that are not bound in their natural state. The fusion protein may be a combination of two, three or even four or more different proteins, including a combination of polypeptides derived from different organism sources, or a combination of polypeptides derived from the same organism source but arranged in a manner different from naturally occurring.

In the present application, the term “vector” refers to a self-replicating DNA molecule that transfer an exogenous gene of interest into a host organism, and is often in the form of a cyclic double-stranded DNA molecule. Typical vectors include plasmids, viruses, phages, cosmids and minichromosomes. Among them, the plasmid is the most common vector form, and refers to a circular double-stranded DNA that can accept an exogenous nucleic acid fragment and can replicate in a prokaryotic or eukaryotic cell.

In the present application, the expressions “expression vector” and “recombinant vector” are used interchangeably, refer to vectors containing exogenous genes, and also comprise regulatory elements expressed in a designated host organism. The introduction of an expression vector into a suitable host organism can enable the host organism to express an inserted gene of interest (for example, the S100A8 fusion gene and the S100A9 fusion gene of the present application).

In the present application, the expression “operably linked” refers to the linking of a plurality of nucleic acid fragments in a functional relationship. When a nucleic acid forms a functional relationship with another nucleic acid sequence, it is “operably linked”. For example, if promoters or other transcriptional regulatory sequences affect the transcription of a coding sequence (or a gene), they are operably linked to the coding sequence in a manner that allows the promoters to induce the transcription of the gene. The “operably linked” means that a linked nucleotide sequence may be continuous or discontinuous.

In the present application, the term “transformation” refers to the transfer of an exogenous gene into a host organism, such as a host cell, resulting in stable genetic inheritance. The transformed gene can be in the form of a plasmid retained in a host organism, or can be integrated into the genome of a host organism. A host organism containing a transformed gene is referred to as a “transgenic” or “recombinant” or “transformed” or “engineered” organism. The transformation of an expression vector into a host organism can be performed using conventional techniques well known to those of ordinary skill in the art. When the host is a prokaryote, competent cells capable of absorbing DNA can be harvested after the logarithmic phase and treated by a CaCl₂) method, and the steps used are well known in the art. If necessary, methods such as microinjection, electroporation or liposome packaging may also be used. These are well-known techniques in the art. which are not described in detail herein.

In the present application, the term “sample” may refer to a biological sample, generally a clinical sample, including blood and other bodily fluids (such as peripheral blood, blood serum, blood plasma, urine and saliva); and solid tissue samples (such as biopsy specimens) are also included. In certain embodiments, the sample comprising blood (such as blood serum or blood plasma) is the most preferred sample type in the present application.

To make the above objectives, features and advantages of the present application more clearly understandable, specific embodiments of the present application are described below in detail.

### Antibody or Antigen-Binding Fragment

According to an embodiment of the present application, an antibody or an antigen-binding fragment thereof that binds to S100A8 or S100A9 is provided, wherein the antibody or the antigen-binding fragment at least comprises one group or more groups selected from groups (i)-(iii) and/or groups (iv)-(v): (i) sequences as shown in SEQ ID NOs: 1, 2, 3, 4 and 6, and RAS; (ii) sequences as shown in SEQ ID NOs: 7, 8, 9, 10 and 12, and EAS; (iii) sequences as shown in SEQ ID NOs: 13, 14, 15, 16 and 18, and RAS; (iv) sequences as shown in SEQ ID NOs: 19, 20, 21, 22 and 24, and AAS; and/or (v) sequences as shown in SEQ ID NOS: 25, 26, 27, 28 and 30, and SAS.

In some embodiments, the antibody or the antigen-binding fragment thereof at least comprises one group or more groups selected from groups (i)-(iii) and/or groups (iv)-(v): (i) a heavy chain variable region (VH) having an amino acid sequence as shown in SEQ ID NO: 31 and a light chain variable region (VL) having an amino acid sequence as shown in SEQ ID NO: 32; (ii) VH having an amino acid sequence as shown in SEQ ID NO: 33 and VL having an amino acid sequence as shown in SEQ ID NO: 34; (iii) VH having an amino acid sequence as shown in SEQ ID NO: 35 and VL having an amino acid sequence as shown in SEQ ID NO: 36; (iv) VH having an amino acid sequence as shown in SEQ ID NO: 37 and VL having an amino acid sequence as shown in SEQ ID NO: 38; (v) VH having an amino acid sequence as shown in SEQ ID NO: 39 and VL having an amino acid sequence as shown in SEQ ID NO: 40; and/or (vi) an amino acid sequence having at least 80%, 85%, 90%, 95%, 98% or 99% identity to VH and/or VL in each of the aforementioned groups.

In some embodiments, the antibody or the antigen-binding fragment thereof further comprises a constant region. In some embodiments, the antibody or the antigen-binding fragment thereof further comprises a signal peptide. In some preferred embodiments, the antibody or the antigen-binding fragment thereof comprises one group or more groups selected from groups (i)-(iii) and/or groups (iv)-(v): (i) a heavy chain having an amino acid sequence as shown in SEQ ID NO: 41 and a light chain having an amino acid sequence as shown in SEQ ID NO: 42; (ii) a heavy chain having an amino acid sequence as shown in SEQ ID NO: 43 and a light chain having an amino acid sequence as shown in SEQ ID NO: 44; (iii) a heavy chain having an amino acid sequence as shown in SEQ ID NO: 45 and a light chain having an amino acid sequence as shown in SEQ ID NO: 46; (iv) a heavy chain having an amino acid sequence as shown in SEQ ID NO: 47 and a light chain having an amino acid sequence as shown in SEQ ID NO: 48; (v) a heavy chain having an amino acid sequence as shown in SEQ ID NO: 49 and a light chain having an amino acid sequence as shown in SEQ ID NO: 50; and/or (vi) an amino acid sequence having at least 80%, 85%, 90%, 95%, 98% or 99% identity to the heavy chain and/or the light chain in each of the aforementioned groups.

According to an embodiment of the present application, a polynucleotide is further provided, wherein the polynucleotide encodes the antibody or the antigen-binding fragment thereof of the present application. In some preferred embodiments, the polynucleotide comprises one group or more groups selected from groups (i)-(iii) and/or groups (iv)-(v): (i) a nucleotide sequence encoding a heavy chain and as shown in SEQ ID NO: 51 and a nucleotide sequence encoding a light chain and as shown in SEQ ID NO: 52; (ii) a nucleotide sequence encoding a heavy chain and as shown in SEQ ID NO: 53 and a nucleotide sequence encoding a light chain and as shown in SEQ ID NO: 54; (iii) a nucleotide sequence encoding a heavy chain and as shown in SEQ ID NO: 55 and a nucleotide sequence encoding a light chain and as shown in SEQ ID NO: 56; (iv) a nucleotide sequence encoding a heavy chain and as shown in SEQ ID NO: 57 and a nucleotide sequence encoding a light chain and as shown in SEQ ID NO: 58; (v) a nucleotide sequence encoding a heavy chain and as shown in SEQ ID NO: 59 and a nucleotide sequence encoding a light chain and as shown in SEQ ID NO: 60; and/or (vi) a nucleotide sequence having at least 80%, 85%, 90%, 95%, 98% or 99% identity to the nucleotide sequence in each of the aforementioned groups.

According to an embodiment of the present application, a recombinant vector is further provided, wherein the recombinant vector comprises the polynucleotide of the present application. In some embodiments, the expression vector is a plasmid vector. In some embodiments, the expression vector is a viral vector. Suitable plasmid vectors and viral vectors are well known in the art.

According to an embodiment of the present application, a host cell is further provided, wherein the host cell comprises the polynucleotide of the present application, and/or the recombinant vector of the present application. The polynucleotide and/or the expression vector may be delivered to a host cell in any suitable manner well known in the art, which is not limited herein.

### Polypeptide, Heterodimer

According to an embodiment of the present application, a polypeptide is further provided, wherein the polypeptide comprises an amino acid sequence having at least 80%, 85%, 90%, 95%, 98% or 99% identity to a sequence as shown in any one of SEQ ID NO: 61, SEQ ID NO: 65, SEQ ID NO: 63 or SEQ ID NO: 67. In some preferred embodiments, the polypeptide comprises a sequence as shown in any one of SEQ ID NO: 61, SEQ ID NO: 65, SEQ ID NO: 63 or SEQ ID NO: 67.

According to an embodiment of the present application, a heterodimer of S100A8 and S100A9 is further provided, wherein the heterodimer comprises amino acid sequences having at least 80%, 85%, 90%, 95%, 98% or 99% identity to sequences as shown in SEQ ID NO: 61 and SEQ ID NO: 67. In some preferred embodiments, the heterodimer comprises sequences as shown in SEQ ID NO: 61 and SEQ ID NO: 67.

According to an embodiment of the present application, a polynucleotide is further provided, wherein the polynucleotide encodes the polypeptide of the present application, or encodes the heterodimer of the present application.

In some embodiments, the polynucleotide comprises a nucleotide sequence having at least 80%, 85%, 90%, 95%, 98% or 99% identity to a sequence as shown in any one of SEQ ID NO: 62, SEQ ID NO: 66, SEQ ID NO: 64 or SEQ ID NO: 68. In some preferred embodiments, the polynucleotide comprises a sequence as shown in any one of SEQ ID NO: 62, SEQ ID NO: 66, SEQ ID NO: 64 or SEQ ID NO: 68.

In some embodiments, the polynucleotide comprises a nucleotide sequence having at least 80%, 85%, 90%, 95%, 98% or 99% identity to sequences as shown in SEQ ID NO: 62 and SEQ ID NO: 68. In some preferred embodiments, the heterodimer comprises sequences as shown in SEQ ID NO: 62 and SEQ ID NO: 68.

According to an embodiment of the present application, an expression vector is further provided, wherein the expression vector comprises the polynucleotide of the present application.

According to an embodiment of the present application, an expression vector encoding the heterodimer of the present application is further provided, wherein the expression vector comprises an S100A8 fusion gene and an S100A9 fusion gene; the S100A8 fusion gene comprises a first promoter and S100A8 gene which are operably linked, and the S100A8 gene has a nucleic acid sequence as shown in SEQ ID NO: 62; and the S100A9 fusion gene comprises a second promoter and S100A9 gene which are operably linked, and the S100A9 gene has a nucleic acid sequence as shown in SEQ ID NO: 68. By inserting S100A8 gene and S100A9 gene into the same vector, and linking the S100A8 gene and the S100A9 gene to 2 promoters respectively, the transcription and translation processes of the S100A8 gene and the S100A9 gene are driven by these 2 promoters respectively, which can ensure that the upstream promoter has less effect on the downstream promoter and basically does not affect the expression of the downstream gene, thereby realizing the co-expression of the S100A8 gene and the S100A9 gene. The promoter can be any suitable promoter sequence, that is, a nucleic acid sequence that can be recognized by a host cell expressing the nucleic acid sequence. The promoter sequence contains a transcriptional regulatory sequence that mediates the expression of the polypeptide and the heterodimer. The promoter can be any nucleic acid sequence having transcriptional activity in a selected host cell, including mutant, truncated and heterozygous promoters, and can be derived from genes encoding extracellular or intracellular proteins or polypeptides homologous or heterologous to the host cell.

In some embodiments, the expression vector comprises an S100A8 fusion gene and an S100A9 fusion gene, the S100A8 fusion gene comprises a first promoter, a ribosome binding site and S100A8 gene which are operably linked from upstream to downstream, and the S100A8 gene has a nucleic acid sequence as shown in SEQ ID NO: 62; and the S100A9 fusion gene comprises a second promoter, a ribosome binding site and S100A9 gene which are operably linked from upstream to downstream, and the S100A9 gene has a nucleic acid sequence as shown in SEQ ID NO: 68. Ribosome binding site (RBS) plays a critical role in the speed and accuracy of protein synthesis. It is used to recognize and bind to ribosomes, which makes mRNA molecules bind more closely to ribosomes. In addition, it can also affect the initiation and termination of the synthesis of a protein of interest, thereby controlling the synthesis speed and quantity of the protein of interest. Appropriate synthesis speed and protein concentration are conducive to promoting the correct folding of a protein, thereby forming a highly active S100A8/S100A9 heterodimer. In some embodiments, the ribosome binding site has a nucleic acid sequence as shown in SEQ ID NO: 71.

In some preferred embodiments, the first promoter and the second promoter are the same. Promoters with the same or similar transcription capability can be selected to make the expression levels of the two genes balanced, that is, the S100A8 recombinant protein and the S100A9 recombinant protein can be secreted in a more balanced manner in a host organism at the same time, thereby ensuring the efficient generation of the heterodimer. In some preferred embodiments, the first promoter and the second promoter are selected from one of a lac promoter, a trp promoter, a tac promoter, a 1pl promoter or a T7 promoter. In some preferred embodiments, the first promoter and the second promoter are T7 promoters, and the T7 promoter has a nucleic acid sequence as shown in SEQ ID NO: 69.

In order to better regulate the expression of S100A8 gene and S100A9 gene, in some embodiments, the S100A8 fusion gene and the S100A9 fusion gene further comprise operators respectively located between the first promoter and the ribosome binding site and between the second promoter and the ribosome binding site. In some preferred embodiments, the operator is a lac operator, and the lac operator has a nucleic acid sequence as shown in SEQ ID NO: 70. That is to say, various genes in the S100A8 fusion gene are sequentially linked according to the following order: T7 promoter, lac operator, RBS and S100A8 gene; and various genes in the S100A9 fusion gene are sequentially linked according to the following order: T7 promoter, lac operator, RBS and S100A9 gene. By using the T7 promoter and the lac operator to control the expression of a gene of interest in a host cell such as a bacterium under the induction of an inducer IPTG, the timing and rate of the expression of the gene of interest can be regulated, which facilitates the correct folding of a protein of interest, achieving soluble expression. At the same time, the soluble S100A8 protein and S100A9 protein synthesized are secreted into the cytoplasm, which can promote the S100A8 protein and S100A9 protein to form an S100A8/S100A9 protein complex.

A stable and soluble expression form is the basis for ensuring the correct folding and biological activity of a protein. In order to further improve the solubility of the S100A8 protein and the S100A9 protein expressed in vitro to better form a heterodimer, in some embodiments, the S100A8 fusion gene and the S100A9 fusion gene further comprise solubilization tags respectively located between the ribosome binding site and the S100A8 gene and between the ribosome binding site and the S100A9 gene. In some embodiments, the solubilization tags include, but are not limited to, maltose-binding protein (MBP) tags, glutathione S-transferase (GST) tags, small ubiquitin-related modifier protein (SUMO) tags, NusA protein (N-utilization substance A) tags, arsenate oxidoreductase (ArsC) tags and T7-tags. The aforementioned tags have little effect on the structure of a protein, and can resist protease hydrolysis, promote the correct folding of a protein of interest, and improve the stability and solubility of the expressed S100A8 protein and S100A9 protein. In some preferred embodiments, the solubilization tags are T7-tags, and the T7 tag has a nucleic acid sequence as shown in SEQ ID NO: 72.

In the in vitro protein expression system of the present application, although the expression amount and purity of the heterodimer (S100A8/S100A9) are greatly improved, a small number of homodimers (S100A8/S100A8 and S100A9/S100A9) and monomer proteins (S100A8 and S100A9) may still exist simultaneously among the proteins expressed by the expression vector. In some embodiments, different purification tags are carried in each of the fusion genes to facilitate isolation and purification without affecting its function. That is to say, the S100A8 fusion gene comprises a first purification tag, the S100A9 fusion gene comprises a second purification tag, the first purification tag and the second purification tag are different. By arranging different purification tags, the first purification tag and the second purification tag can be used sequentially to perform purification, so as to effectively isolate the S100A8/S100A9 protein from the aforementioned multi-protein system. Specifically, the first purification tag is used first to perform purification, so as to isolate the S100A8 and S100A8/S100A9 having the first purification tag, and then the second purification tag is used to perform purification to further isolate the S100A8/S100A9, so as to obtain the heterodimer with high purity and high concentration. Those of ordinary skill in the art can understand that the second purification tag can also be used first to perform purification, and then the first purification tag is used to perform purification. In some embodiments, the purification tag is selected from one or more of 6xHis tag, 8xHis tag, Trx tag, Flag tag, strep (II) tag, HA tag, GFP tag, cMyc tag and mFC tag. Those of ordinary skill in the art can make specific arrangement according to the purification requirements to be suitable for different purification systems and equipment, which is not limited in the present application. In some preferred embodiments, the first purification tag is a Flag tag, and the Flag tag is located upstream of the S100A8 gene, and has a nucleic acid sequence as shown in SEQ ID NO: 73. The second purification tag is a 6xHis tag, and the 6xHis tag is located downstream of the S100A9 gene, and has a nucleic acid sequence as shown in SEQ ID NO: 74. With the aforementioned arrangement, the secreted S100A8 protein carries a Flag tag at the N-terminus, while the S100A9 protein carries a 6xHis tag at the C-terminus. Therefore, a protein complex of interest can be obtained by His affinity purification and Flag affinity purification.

In some embodiments, the expression vector is a plasmid vector. In some embodiments, the expression vector is a viral vector. Suitable plasmid vectors and viral vectors are well known in the art. In some preferred embodiments, the expression vector is a prokaryotic expression vector. By sequentially linking a T7 promoter, a lac operator, a ribosome binding site, a T7-tag, a Flag tag and S100A8 gene to form an S100A8 fusion gene, sequentially linking a T7 promoter, a lac operator, a ribosome binding site, a T7-tag, S100A9 gene and a 6xHis tag to form an S100A9 fusion gene, and linking the S100A8 fusion gene and the S100A9 fusion gene to the multiple cloning sites of a prokaryotic expression vector, the expression vector of the present application can be obtained. That is to say, in the expression vector, various genes in the S100A8 fusion gene are sequentially linked according to the following order: T7 promoter, lac operator, RBS, T7-tag, Flag tag and S100A8 gene; and various genes in the S100A9 fusion gene are sequentially linked according to the following order: T7 promoter, lac operator, RBS, T7-tag, S100A9 gene and 6xHis tag. By transforming the expression vector into a prokaryotic host cell, S100A8 recombinant protein and S100A9 recombinant protein are co-expressed, wherein the S100A8 recombinant protein, from N-terminus to C-terminus, sequentially comprises: T7-tag, Flag tag and S100A8 protein, and the S100A9 recombinant protein, from N-terminus to C-terminus, sequentially comprises: T7-tag, S100A9 protein and 6×His tag.

In some preferred embodiments, the expression vector is pET-28a. Since the pET-28a carries T7 promoter, lac operator, ribosome binding site and T7-tag, when the expression vector of the present application is constructed, only Flag tag. S100A8 gene and S100A9 fusion gene need to be inserted after the corresponding site in the starting vector to form the above-mentioned S100A8 fusion gene and S100A9 fusion gene, thereby simplifying the construction process.

According to an embodiment of the present application, a host cell is further provided, wherein the host cell comprises the polynucleotide of the present application, and/or the expression vector of the present application. The host cell can be selected according to the type of the expression vector. In some preferred embodiments, the expression vector is a prokaryotic expression vector, and the host cell is a prokaryotic cell. Examples of common prokaryotic host cells include Escherichia coli, etc.

The polynucleotide and/or the expression vector may be delivered to a host cell in any suitable manner well known in the art, which is not limited herein. After the host cell comprising the expression vector is obtained, the host cell is cultured under suitable conditions to co-express the S100A8 recombinant protein and the S100A9 recombinant protein, and then affinity purification is performed sequentially through the purification tags contained in each recombinant protein to obtain the S100A8/S100A9 heterodimer with high purity and high concentration.

### Kit

According to an embodiment of the present application, a kit is further provided, wherein the kit comprises the antibody or the antigen-binding fragment thereof of the present application, the polypeptide of the present application, and/or the heterodimer of the present application.

In some embodiments, the kit is used to detect S100A8, S100A9, and/or a dimer of S100A8 and S100A9 in a sample by immunoassay.

The kit can use any suitable immunological detection method known in the art to detect S100A8, S100A9, and/or a S100A8/S100A9 dimer in a sample, non-limiting examples of the method include an enzyme linked immunosorbent assay (ELISA) method, an immunoblotting method, an immunofluorescence method, a method, an immunohistochemistry radioimmunoassay method, an immunocolloidal gold technique, or Point of Care Testing (POCT). In some preferred embodiments, the immunoassay is ELISA.

The sample can be any suitable sample from a subject. In some embodiments, the sample includes, but is not limited to, blood, tissue, cells, body fluids, urine, or fecal extracts. In some preferred embodiments, the sample is blood.

The detection objects of the kit include S100A8, S100A9 and an S100A8/S100A9 dimer, that is, the kit can not only detect an S100A8/S100A9 heterodimer, but also can only detect S100A8 or only detect S100A9, as long as the corresponding antibody and/or standard are selected. In some embodiments, the kit comprises an anti-S100A8 antibody and an S100A8 polypeptide, and can be used for detecting S100A8 in a sample. In some embodiments, the kit comprises an anti-S100A9 antibody and an S100A9 polypeptide, and can be used for detecting S100A9 in a sample. In some embodiments, the kit comprises an anti-S100A8 antibody and/or an anti-S100A9 antibody, and further comprises an S100A8/S100A9 heterodimer, and can be used for detecting an S100A8/S100A9 heterodimer in a sample.

In some preferred embodiments, the kit is a double antibody sandwich ELISA kit, and can be used for detecting an S100A8/S100A9 heterodimer in a liquid sample. The kit uses the antibody pairing strategy, that is, the capture antibody is an anti-S100A8 antibody and the labeled antibody is an anti-S100A9 antibody, or the capture antibody is an anti-S100A9 antibody and the labeled antibody is an anti-S100A8 antibody. Moreover, the standard in the kit is the S100A8/S100A9 heterodimer provided by the present application.

The anti-S100A8 antibody can be used as the capture antibody and the anti-S100A9 antibody can be used as the labeled antibody. In some embodiments, the capture antibody is selected from one group or more groups of groups (i)-(iii), and/or the labeled antibody is selected from one group or more groups of groups (iv)-(v): (i) VH having an amino acid sequence as shown in SEQ ID NO: 31 and VL having an amino acid sequence as shown in SEQ ID NO: 32; (ii) VH having an amino acid sequence as shown in SEQ ID NO: 33 and VL having an amino acid sequence as shown in SEQ ID NO: 34; (iii) VH having an amino acid sequence as shown in SEQ ID NO: 35 and VL having an amino acid sequence as shown in SEQ ID NO: 36; (iv) VH having an amino acid sequence as shown in SEQ ID NO: 37 and VL having an amino acid sequence as shown in SEQ ID NO: 38; and/or (v) VH having an amino acid sequence as shown in SEQ ID NO: 39 and VL having an amino acid sequence as shown in SEQ ID NO: 40.

In some preferred embodiments, the VH of the capture antibody has a sequence as shown in SEQ ID NO: 31, the VL of the capture antibody has a sequence as shown in SEQ ID NO: 32, the VH of the labeled antibody has a sequence as shown in SEQ ID NO: 37, and the VL of the labeled antibody has a sequence as shown in SEQ ID NO: 38; the VH of the capture antibody has a sequence as shown in SEQ ID NO: 31, the VL of the capture antibody has a sequence as shown in SEQ ID NO: 32, the VH of the labeled antibody has a sequence as shown in SEQ ID NO: 39, and the VL of the labeled antibody has a sequence as shown in SEQ ID NO: 40; the VH of the capture antibody has a sequence as shown in SEQ ID NO: 33, the VL of the capture antibody has a sequence as shown in SEQ ID NO: 34, the VH of the labeled antibody has a sequence as shown in SEQ ID NO: 37, and the VL of the labeled antibody has a sequence as shown in SEQ ID NO: 38; the VH of the capture antibody has a sequence as shown in SEQ ID NO: 35, the VL of the capture antibody has a sequence as shown in SEQ ID NO: 36, the VH of the labeled antibody has a sequence as shown in SEQ ID NO: 37, and the VL of the labeled antibody has a sequence as shown in SEQ ID NO: 38; or the VH of the capture antibody has a sequence as shown in SEQ ID NO: 35, the VL of the capture antibody has a sequence as shown in SEQ ID NO: 36, the VH of the labeled antibody has a sequence as shown in SEQ ID NO: 39, and the VL of the labeled antibody has a sequence as shown in SEQ ID NO: 40.

Alternatively, the anti-S100A9 antibody can be used as the capture antibody and the anti-S100A8 antibody can be used as the labeled antibody. In some embodiments, the labeled antibody is selected from one group or more groups of groups (i)-(iii), and/or the capture antibody is selected from one group or more groups of groups (iv)-(v): (i) VH having an amino acid sequence as shown in SEQ ID NO: 31 and VL having an amino acid sequence as shown in SEQ ID NO: 32; (ii) VH having an amino acid sequence as shown in SEQ ID NO: 33 and VL having an amino acid sequence as shown in SEQ ID NO: 34; (iii) VH having an amino acid sequence as shown in SEQ ID NO: 35 and VL having an amino acid sequence as shown in SEQ ID NO: 36; (iv) VH having an amino acid sequence as shown in SEQ ID NO: 37 and VL having an amino acid sequence as shown in SEQ ID NO: 38; and/or (v) VH having an amino acid sequence as shown in SEQ ID NO: 39 and VL having an amino acid sequence as shown in SEQ ID NO: 40.

In some preferred embodiments, the VH of the capture antibody has a sequence as shown in SEQ ID NO: 37, the VL of the capture antibody has a sequence as shown in SEQ ID NO: 38, the VH of the labeled antibody has a sequence as shown in SEQ ID NO: 31, and the VL of the labeled antibody has a sequence as shown in SEQ ID NO: 32; the VH of the capture antibody has a sequence as shown in SEQ ID NO: 37, the VL of the capture antibody has a sequence as shown in SEQ ID NO: 38, the VH of the labeled antibody has a sequence as shown in SEQ ID NO: 35, and the VL of the labeled antibody has a sequence as shown in SEQ ID NO: 36; the VH of the capture antibody has a sequence as shown in SEQ ID NO: 39, the VL of the capture antibody has a sequence as shown in SEQ ID NO: 40, the VH of the labeled antibody has a sequence as shown in SEQ ID NO: 31, and the VL of the labeled antibody has a sequence as shown in SEQ ID NO: 32; the VH of the capture antibody has a sequence as shown in SEQ ID NO: 39, the VL of the capture antibody has a sequence as shown in SEQ ID NO: 40, the VH of the labeled antibody has a sequence as shown in SEQ ID NO: 35, and the VL of the labeled antibody has a sequence as shown in SEQ ID NO: 36; or the VH of the capture antibody has a sequence as shown in SEQ ID NO: 37, the VL of the capture antibody has a sequence as shown in SEQ ID NO: 38, the VH of the labeled antibody has a sequence as shown in SEQ ID NO: 33, and the VL of the labeled antibody has a sequence as shown in SEQ ID NO: 34.

In some embodiments, the labeled antibody has a detectable label to facilitate quantitative detection. The detectable label can be any suitable label known in the art, including but not limited to enzymes, chemiluminescent reagents, fluorescent dyes, colloidal gold or biotin. In some preferred embodiments, the detectable label is biotin.

### Use

According to an embodiment of the present application, the use of the antibody or the antigen-binding fragment thereof of the present application, and/or the polypeptide of the present application in the preparation of a reagent, a kit, an antibody chip or an antibody probe for detecting S100A8, S100A9, and a dimer of S100A8 and S100A9 is further provided.

According to an embodiment of the present application, the use of the heterodimer of the present application in the preparation of a reagent and a kit for detecting a dimer of S100A8 and S100A9 is further provided.

According to an embodiment of the present application, the use of the antibody or the antigen-binding fragment thereof of the present application, the polypeptide of the present application, and/or the heterodimer of the present application in the preparation of a reagent, a kit, an antibody chip or an antibody probe for detecting a disease or a non-disease symptom. In some embodiments, the disease or the non-disease symptom is associated with a change in the expression level of the dimer of S100A8 and S100A9.

In some embodiments, the disease or the non-disease symptom is associated with an increase in the expression level of the dimer of S100A8 and S100A9. Within the range known in the art, there are a variety of diseases or non-disease symptoms accompanied by an increase in the expression level of the S100A8/S100A9 dimer. The disease or the non-disease symptom includes, but are not limited to: sepsis (Am J Respir Crit Care Med, 2017, 196 (3): 315-327), an autoimmune disease (Autoimmun Rev, 2023, 22 (5): 103295), acute lung injury (Blood, 2022, 140 (24): 2626-2643), acute pancreatitis (Dis Markers, 2018, 2018:6457347), acute kidney injury (Adv Sci (Weinh), 2022, 9 (12): e2103675), tumor (Sci Transl Med, 2020, 12 (572): cabb5817), organ ischemia/reperfusion injury (Circulation, 2019, 140 (9): 751-764; Kidney Int. 2015, 87 (1): 85-94), and an atherosclerosis-associated cardio-cerebral disease (Pharmacol Res, 2020, 161:105212), the disclosure of which is incorporated herein by reference in its entirety. Therefore, the reagent, kit, antibody chip or antibody probe can be used for detecting the expression level of the S100A8/S100A9 dimer in a sample to judge the risk of a disease or a non-disease symptom.

In some preferred embodiments, the disease or the non-disease symptom is selected from sepsis, an autoimmune disease (such as systemic lupus erythematosus and ankylosing spondylitis), acute lung injury (such as chronic obstructive pulmonary disease and viral pneumonia), acute pancreatitis, acute kidney injury, tumors (such as pancreatic cancer), organ ischemia/reperfusion injury (such as myocardial ischemia/reperfusion injury, renal ischemia/reperfusion injury, liver ischemia/reperfusion injury and cerebral ischemia/reperfusion injury), and an atherosclerosis-associated cardio-cerebral disease (such as coronary heart disease, cerebral infarction and myocardial infarction).

### Disease Diagnosis Method

According to an embodiment of the present application, a method for detecting a disease or a non-disease symptom is further provided, wherein the method comprises the following steps: obtaining a sample of a subject; using the antibody or the antigen-binding fragment thereof of the present application, the polypeptide of the present application, the heterodimer of the present application, and/or the kit of the present application to detect the expression level of the dimer of S100A8 and S100A9 in the sample; and judging that the subject has the disease or the non-disease symptom according to the expression level of the dimer of S100A8 and S100A9 in the sample.

The sample can be any suitable sample from a subject. In some embodiments, the sample is blood, tissue, cells, body fluids, urine, or fecal extracts. In some preferred embodiments, the sample is blood.

In some embodiments, the disease or the non-disease symptom is associated with an increase in the expression level of the dimer of S100A8 and S100A9, preferably, wherein the disease or the non-disease symptom is selected from sepsis, an autoimmune disease (such as systemic lupus erythematosus and ankylosing spondylitis), acute lung injury (such as chronic obstructive pulmonary disease and viral pneumonia), acute pancreatitis, acute kidney injury, tumors (such as pancreatic cancer), organ ischemia/reperfusion injury (such as myocardial ischemia/reperfusion injury, renal ischemia/reperfusion injury, liver ischemia/reperfusion injury and cerebral ischemia/reperfusion injury), and an atherosclerosis-associated cardio-cerebral disease (such as coronary heart disease, cerebral infarction and myocardial infarction).

The above-mentioned various embodiments and preferences in the present disclosure can be combined with each other (as long as they are not inherently contradictory to each other), and the various embodiments formed by the combination are considered as a part of the disclosure of the present application.

Exemplary examples of the present application are described below in conjunction with the accompanying drawings, where various details of the examples of the present application are included to facilitate understanding. It should be understood that they are considered to be exemplary only and not intended to limit the protection scope of the present application. The protection scope of the present application is only defined by the claims. Therefore, those of ordinary skill in the art should be aware that various changes and modifications can be made to the examples described herein, without departing from the scope of the present application. Likewise, for clarity and conciseness, the description of well-known functions and structures is omitted in the following description.

### EXAMPLES

If specific techniques or conditions are not specified in the examples, techniques or conditions described in the literature in the art or techniques or conditions according to the product instructions are followed. The reagents or instruments used therein for which manufacturers are not specified are all conventional products that are commercially available.

### Example 1: Preparation of S100A8 Polypeptide, S100A9 Polypeptide and Heterodimer of S100A8 and S100A9

### 1.1 Construction of Expression Vectors 1-4 (Plasmids 1-4)

### (1) Selection of Mutation Sites

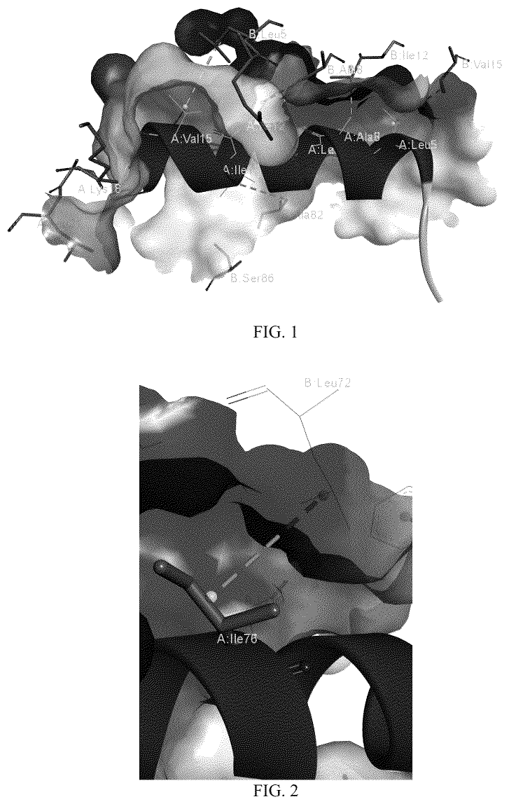

The interaction between the human S100A8s in the homodimer (NCBI accession number: NM_001319201.1) was analyzed through the resolved crystal structure (1MR8). It was found that the human S100A8s in the homodimer mainly interacted with each other through hydrophobic interaction, mainly at N-terminus 5-17 aa and 68-86 aa, wherein at N-terminus 5-17 aa, hydrophobic interaction existed between A chain A8 and B chain 112, between A chain 112 and B chain A8, between A chain 113 and B chain A82, and between A chain L9 and B chain M78 (the hydrophobic interaction between A chain L9 and B chain M78 was relatively weak and can be negligible), and at N-terminus 68-86 aa, hydrophobic interaction mainly existed between A chain 178 and B chain L72. Therefore, in the present application, the three sites 112, 113 and L72 were selected to mutate into alanine A (the mutation sequence is hereinafter referred to as S100A8-1, and the amino acid sequence is as shown in SEQ ID NO: 65), so as to reduce the hydrophobic interaction between the S100A8s in the homodimer to reduce the formation of homodimers. FIG. 1 and FIG. 2 show schematic diagrams of the interaction between the human S100A8s in the homodimer, in which FIG. 1 illustrates the two mutation sites 112 and 113, FIG. 2 illustrates the mutation site L72, and the dotted line indicates the hydrophobic interaction.

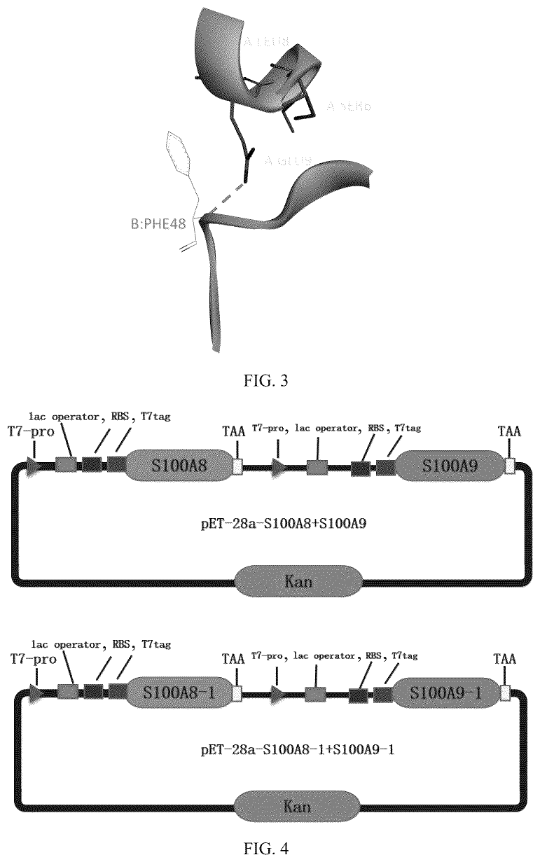

The human S100A9s in the homodimer (NCBI accession number: NM_002965.3) also mainly interacted with each other by hydrophobic interaction, and there was also a hydrogen bonding force between A chain E9 and B chain F48. Therefore, hydrophilic acidic amino acid E9 was selected to mutate into hydrophobic alanine A (the mutation sequence is hereinafter referred to as S100A9-1, and the amino acid sequence is as shown in SEQ ID NO: 67). FIG. 3 shows a schematic diagram of the interaction between the human S100A9s in the homodimer, in which two monomer molecules of the homodimer were shown, and the dotted line indicates the hydrophobic interaction.

### (2) Construction of Expression Vectors

The information of the expression vectors constructed in this example and the sequence involved were as shown in Table 1 and Table 2. The vector was pET-28a. FIG. 4 shows the vector maps of plasmid 3 (upper panel) and plasmid 4 (lower panel).

TABLE 1
Structure of expression vector

| Vector name | Vector structure |
|---|---|
| Plasmid 1 | Flag tag - S100A8 |
| Plasmid 2 | S100A9 - 6 × His tag |
| Mutant plasmid 1 | Flag tag - S100A8-1 (I12A, I13A, L72A) |
| Mutant plasmid 2 | S100A9-1 (E9A) - 6 × His tag |
| Plasmid 3 | Flag tag, S100A8 gene, T7 promoter, lac operator, ribosome binding site, |
| | T7-tag, S100A9 gene and 6 × His tag (hereafter referred to as |
| | S100A8 + S100A9) |
| Plasmid 4 | Flag tag, S100A8-1 gene, T7 promoter, lac operator, ribosome binding site, |
| | T7-tag, S100A9-1 gene and 6 × His tag (hereafter referred to as |
| | S100A8-1 + S100A9-1) |
| Plasmid 5 | Flag tag, S100A8 gene, T7 promoter, lac operator, ribosome binding site, |
| | T7-tag, S100A9-1 gene and 6 × His tag (hereafter referred to as |
| | S100A8 + S100A9-1) |

Genes S100A8, S100A9, S100A8-1 (I12A, 113A, L72A) and S100A9-1 (E9A) (see SEQ ID NOs: 61-68) were synthesized by a whole gene synthesis method, and Flag tag and 6xHis tag (see SEQ ID NOs: 75-77 for purification tag insertion site) were added to the genes S100A8 and S100A9 respectively. These synthesized genes were then constructed into pET-28a vector at the insertion site BamHI/HindIII. The enzyme cleavage and enzyme ligation operations were performed using conventional techniques in the art. The successfully constructed recombinant vectors were named as plasmid 1, plasmid 2, mutant plasmid 1 and mutant plasmid 2 respectively.

Similarly, using a whole gene synthesis method, S100A8 sequence having Flag tag at the N-terminus and S100A9 sequence having His tag at the C-terminus were ligated via T7 promoter, lac operator, ribosome binding site and T7-tag therebetween, and then constructed into pET-28a vector at the insertion site Ncol/HindIII to obtain plasmid 3 for gene co-expression. S100A8-1 sequence having Flag tag at the N-terminus and S100A9-1 sequence having his tag at the C-terminus were ligated via T7 promoter, lac operator, ribosome binding site and T7-tag therebetween, and then constructed into pET-28a vector at the insertion site Ncol/HindIII to obtain plasmid 4 for gene co-expression.

Since the pET-28a carries a T7 promoter (T7-pro), a lac operator, a ribosome binding site and a T7-tag, when the expression vector of the present application is constructed, only Flag tag, S100A8 gene and S100A9 fusion gene (T7 promoter, lac operator, ribosome binding site, T7-tag, S100A9 gene and 6xHis tag) need to be inserted after the corresponding sites in the vector to simplify the construction process of the expression vector. It should be noted that when the vector is not pET-28a, the inserted gene of interest is the sequence

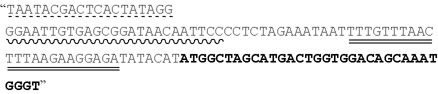

(the underlined dashed line represents T7 promoter, the underlined wavy line represents lac operator, the underlined double line represents ribosome binding site, and the bolded section represents T7-tag) inserted upstream of the corresponding sequence, for example, when plasmid 5 is constructed, this sequence is inserted upstream of SEQ ID NO: 77.

TABLE 2
Sequence of expression vector

| Name | Type | Sequence |
|---|---|---|
| S100A8 | Amino | SEQ ID NO: 61 |
| | acid | |
| | DNA | SEQ ID NO: 62 |
| S100A9 | Amino | SEQ ID NO: 63 |
| | acid | |
| | DNA | SEQ ID NO: 64 |
| S100A8-1 | Amino | SEQ ID NO: 65 (mutation sites I12A, I13A and L72A) |
| | acid | |
| | DNA | SEQ ID NO: 66 |
| S100A9-1 | Amino | SEQ ID NO: 67 (mutation site E9A) |
| | acid | |
| | DNA | SEQ ID NO: 68 |
| T7-pro | DNA | SEQ ID NO: 69 |
| lac | DNA | SEQ ID NO: 70 |
| RBS | DNA | SEQ ID NO: 71 |
| T7-tag | DNA | SEQ ID NO: 72 |
| Flag | DNA | SEQ ID NO: 73 |
| 6 × His | DNA | SEQ ID NO: 74 |
| Plasmid 3 | DNA | ATGGATTACAAGGACGACGATGACAAGTTGACCGAGCTGGA |
| | | GAAAGCCTTGAACTCTATCATCGACGTCTACCACAAGTACT |
| | | CCCTGATAAAGGGGAATTTCCATGCCGTCTACAGGGATGAC |
| | | CTGAAGAAATTGCTAGAGACCGAGTGTCCTCAGTATATCAG |
| | | GAAAAAGGGTGCAGACGTCTGGTTCAAAGAGTTGGATATCA |
| | | ACACTGATGGTGCAGTTAACTTCCAGGAGTTCCTCATTCTGG |
| | | TGATAAAGATGGGCGTGGCAGCCCACAAAAAAAGCCATGA |
| | | 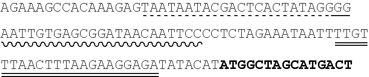 |
| | | GGTGGACAGCAAATGGGTATGACTTGCAAAATGTCGCAGC |
| | | TGGAACGCAACATAGAGACCATCATCAACACCTTCCACCAA |
| | | TACTCTGTGAAGCTGGGGCACCCAGACACCCTGAACCAGGG |
| | | GGAATTCAAAGAGCTGGTGCGAAAAGATCTGCAAAATTTTC |
| | | TCAAGAAGGAGAATAAGAATGAAAAGGTCATAGAACACAT |
| | | CATGGAGGACCTGGACACAAATGCAGACAAGCAGCTGAGC |
| | | TTCGAGGAGTTCATCATGCTGATGGCGAGGCTAACCTGGGC |
| | | CTCCCACGAGAAGATGCACGAGGGTGACGAGGGCCCTGGCC |
| | | ACCACCATAAGCCAGGCCTCGGGGAGGGCACCCCCCACCAC |
| | | CACCACCACCACTAA (see SEQ ID NO: 75), wherein the underlined |
| | | single line represents Flag tag, the underlined dashed line represents |
| | | T7 promoter, the underlined wavy line represents lac operator, the |
| | | underlined double line represents ribosome binding site, the bolded |
| | | section represents T7-tag, and the italicized section represents 6 × His |
| | | tag |
| Plasmid 4 | DNA | ATGGATTACAAGGACGACGATGACAAGTTGACCGAGCTGGA |
| | | GAAAGCCTTGAACTCTGCCGCCGACGTCTACCACAAGTACT |
| | | CCCTGATAAAGGGGAATTTCCATGCCGTCTACAGGGATGAC |
| | | CTGAAGAAATTGCTAGAGACCGAGTGTCCTCAGTATATCAG |
| | | GAAAAAGGGTGCAGACGTCTGGTTCAAAGAGTTGGATATCA |
| | | ACACTGATGGTGCAGTTAACTTCCAGGAGTTCGCCATTCTGG |
| | | TGATAAAGATGGGCGTGGCAGCCCACAAAAAAAGCCATGA |
| | | 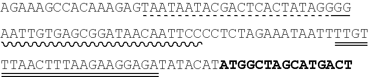 |
| | | GGTGGACAGCAAATGGGTATGACTTGCAAAATGTCGCAGC |
| | | TGGCACGCAACATAGAGACCATCATCAACACCTTCCACCAA |
| | | TACTCTGTGAAGCTGGGGCACCCAGACACCCTGAACCAGGG |
| | | GGAATTCAAAGAGCTGGTGCGAAAAGATCTGCAAAATTTTC |
| | | TCAAGAAGGAGAATAAGAATGAAAAGGTCATAGAACACAT |
| | | CATGGAGGACCTGGACACAAATGCAGACAAGCAGCTGAGC |
| | | TTCGAGGAGTTCATCATGCTGATGGCGAGGCTAACCTGGGC |
| | | CTCCCACGAGAAGATGCACGAGGGTGACGAGGGCCCTGGCC |
| | | ACCACCATAAGCCAGGCCTCGGGGAGGGCACCCCCCACCAC |
| | | CACCACCACCACTAA (see SEQ ID NO: 76), wherein the underlined |
| | | single line represents Flag tag, the underlined dashed line represents |
| | | T7 promoter, the underlined wavy line represents lac operator, the |
| | | underlined double line represents ribosome binding site, the bolded |
| | | section represents T7-tag, and the italicized section represents 6 × His |
| | | tag |
| Plasmid 5 | DNA | ATGGATTACAAGGACGACGATGACAAGTTGACCGAGCTGGA |
| | | GAAAGCCTTGAACTCTATCATCGACGTCTACCACAAGTACT |
| | | CCCTGATAAAGGGGAATTTCCATGCCGTCTACAGGGATGAC |
| | | CTGAAGAAATTGCTAGAGACCGAGTGTCCTCAGTATATCAG |
| | | GAAAAAGGGTGCAGACGTCTGGTTCAAAGAGTTGGATATCA |
| | | ACACTGATGGTGCAGTTAACTTCCAGGAGTTCCTCATTCTGG |
| | | TGATAAAGATGGGCGTGGCAGCCCACAAAAAAAGCCATGA |
| | | 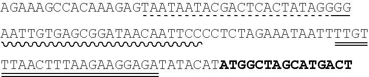 |
| | | GGTGGACAGCAAATGGGTATGACTTGCAAAATGTCGCAGC |
| | | TGGCACGCAACATAGAGACCATCATCAACACCTTCCACCAA |
| | | TACTCTGTGAAGCTGGGGCACCCAGACACCCTGAACCAGGG |
| | | GGAATTCAAAGAGCTGGTGCGAAAAGATCTGCAAAATTTTC |
| | | TCAAGAAGGAGAATAAGAATGAAAAGGTCATAGAACACAT |
| | | CATGGAGGACCTGGACACAAATGCAGACAAGCAGCTGAGC |
| | | TTCGAGGAGTTCATCATGCTGATGGCGAGGCTAACCTGGGC |
| | | CTCCCACGAGAAGATGCACGAGGGTGACGAGGGCCCTGGCC |
| | | ACCACCATAAGCCAGGCCTCGGGGAGGGCACCCCCCACCAC |
| | | CACCACCACCACTAA (see SEQ ID NO: 77), wherein the underlined |
| | | single line represents Flag tag, the underlined dashed line represents |
| | | T7 promoter, the underlined wavy line represents lac operator, the |
| | | underlined double line represents ribosome binding site, the bolded |
| | | section represents T7-tag, and the italicized section represents 6 × His |
| | | tag |

### 1.2 Expression and Purification of Expression Vectors 1-4 (Plasmids 1-4)

(1) Transformation into host bacteria: the above expression vectors were respectively transformed into Escherichia coli BL21 (DE3), and positive clones were screened by conventional techniques;

(2) Induction of bacterial liquid: the transformed positive clones were picked and inoculated into LB medium, the inoculated medium was placed in a shaker for culturing 2-3 h at 37° C. and 220 rpm, and when OD600 reached about 0.5-0.55, the culture was induced with 0.2 mM isopropyl thiogalactoside (IPTG) (purchased from Biosharp, Cat. No. BS119) at 16° C. and 180 rpm for 16 h;

(3) Collection of bacteria: the bacteria liquid after the induction was collected into a centrifugal bottle, centrifuged at 4000 rpm, 4° C. for 10 min by a high speed centrifuge, the supernatant was discarded, and the bacteria were collected;

(4) Ultrasonication: 30 mL of a lysis solution (comprising 50 mM Tris-HCl and 300 mM NaCl/500 mM NaCl) was taken for suspending the bacteria in a centrifugal bottle, and then ultrasonication was performed for 5 min at an ultrasonic power of 350 W, an ultrasonication time of 3 s and an interval between ultrasonication of 3 s, then the bottle was then placed in an ice-water mixture for cooling for 5 min, and above steps were then repeated for lysing for 5 min;

(5) Collection of supernatant: the supernatant after lysis was collected by centrifugation on a high speed centrifuge at 9000 rpm, 4° C. for 10 min;

(6) Purification of His-Tag fusion protein: the supernatant obtained after lysis and centrifugation was mixed with NiBestarose6FF matrix (purchased from Bestchrom (Shanghai), Cat. No. AA0052) in a 50 mL centrifugal tube and bound for 45 min in a mixing incubator at 4° C., the liquid after the binding was added to a pre-packed column and eluted with Washing Buffer at a controlled flow rate of 2 mL/min, and the flow-through was collected; and subsequently, the protein of interest was eluted with Elution Buffers A and B in turn, and Elution Buffers A and B were collected in a 10 mL centrifugal tube. The formulas of buffers for purifying His-Tag fusion protein are as shown in Table 3.

TABLE 3
Formulas of buffers for purifying His-Tag fusion protein

| | Tris | NaCl | Imidazole | pH |
|---|---|---|---|---|
| Buffer name | (mM) | (mM) | (mM) | value |
| Binding Buffer | 50 | 250 | 0 | pH 8.0 |
| Washing Buffer | 50 | 250 | 50 | pH 8.0 |
| Elution Buffer A | 50 | 250 | 250 | pH 8.0 |
| Elution Buffer B | 50 | 250 | 500 | pH 8.0 |

(7) Purification of Flag-Tag fusion protein: a) Anti-DYKDDDDK Affinity Resin (purchased from Genscript (Nanjing) Co., Ltd., Cat. No. L00766) was washed with 10-20 mL of TBS buffer to reduce non-specific adsorption; b) the prepared resin and the above-mentioned eluent sample after His purification (the proteins after the Elution Buffers A and B were mixed) were completely re-suspended in a chromatographic column, and bound at 4° C. for 16 h on a mixing incubator, and the flow-through was collected under gravity flow conditions; c) the resin was washed with 10-20 mL of TBS buffer to remove any non-specific binding; d) the protein of interest was eluted with 3 mL of Elution Buffer A, and the eluent was collected in a 2 mL centrifugal tube at a flow rate under gravity at 1 mL/tube, with a total of 3 tubes; and e) the resin was rinsed with 2 mL of Elution Buffer B to isolate the Flag polypeptide bound to the resin, the liquid flowed out at a flow rate under gravity, Elution Buffer B was collected, and Elution Buffers A and B were mixed to obtain the purified protein of interest. The formulas of buffers for purifying Flag-Tag fusion protein are as shown in Table 4.

TABLE 4
Formulas of buffers for purifying Flag-Tag fusion protein

| | Tris | NaCl | Flag polypeptide | pH |
|---|---|---|---|---|
| Buffer name | (mM) | (mM) | (mM) | value |
| TBS | 50 | 150 | 0 | pH 7.4 |
| Elution Buffer A | 50 | 150 | 500 | pH 7.4 |
| Elution Buffer B | 100 | 500 | 0 | pH 12.0 |

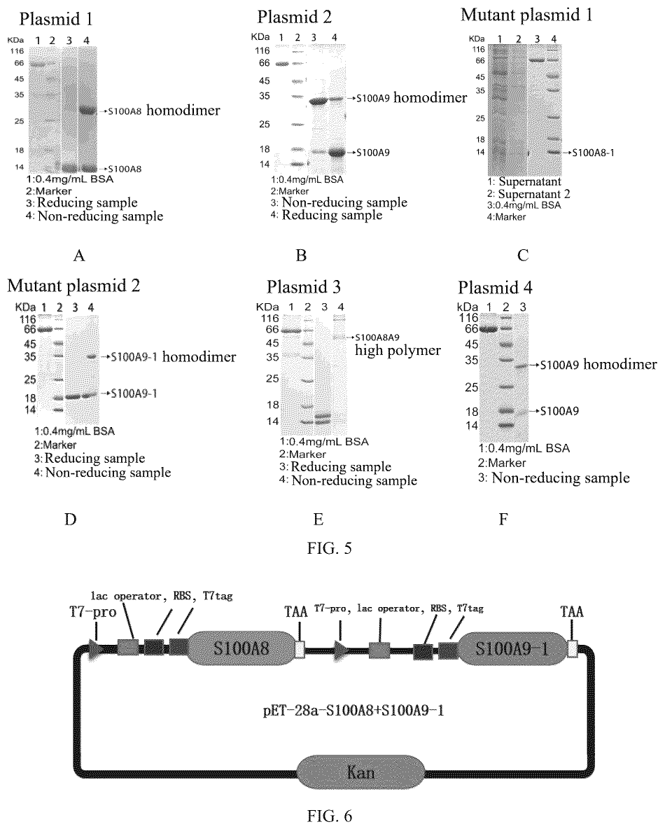

(8) SDS-PAGE gel electrophoresis: the supernatant obtained after lysis and the purified protein were mixed with 6×Loading Buffer according to a volume ratio of 5:1 for sample preparation; and the prepared sample was subjected to SDS-PAGE gel electrophoresis together with bovine serum albumin (BSA, purchased from BioFroxx, Cat. No. 4240GR025) and Marker, wherein plasmid 1 and mutant plasmid 1 were the final samples purified by Flag-Tag, plasmid 2 and mutant plasmid 2 were the final samples purified by His-Tag, and plasmid 3 and plasmid 4 were the final samples purified by His-Tag and Flag-Tag. The electrophoresis results are as shown in FIG. 5, where panels A-F represent the SDS-PAGE electrophoretograms of the expressed and purified proteins from plasmid 1, plasmid 2, mutant plasmid 1, mutant plasmid 2, plasmid 3 and plasmid 4, respectively, in which in the reducing sample, DTT is added, while in the non-reducing sample, DTT is not added. In panel C, the “Supernatant” refers to the supernatant obtained after centrifugation during first lysis, and the “Supernatant 2” refers to the supernatant obtained by performing second lysis treatment on the precipitate obtained after first lysis and centrifugation and then performing centrifugation.

S100A8 has a theoretical size of 15 KDa, and has a homodimer size of 30 KDa, and S100A9 has a theoretical size of 17 KDa, and has a homodimer size of 34 KDa. It can be seen from the figures that relatively more S100A8 homodimers are present in the expression product from plasmid 1, and by comparing with the concentration of BSA (0.4 mg/mL) through the band size in the gel image, it can be seen that the S100A8 homodimer at the size of 30 KDa has a protein concentration of 1 mg/mL (see panel A). Relatively more S100A9 homodimers are present in the expression product from plasmid 2, and by comparing with the concentration of BSA (0.4 mg/mL) through the band size in the gel image, it can be seen that the S100A9 homodimer at the size of 34 KDa has a protein concentration of 0.3 mg/mL (see panel B). No obvious protein at a size of 15 KDa is observed in the expression product from mutant plasmid 1, indicating that the mutation sites such as I12A, 113A and L72A of S100A8 do not increase soluble expression, and a homodimer cannot be formed (see panel C). An S100A9 homodimer is present in the expression product from mutant plasmid 2, and is obviously decreased compared with that in the expression product from plasmid 2 (see panel D). In the expression product from plasmid 3, homologous proteins of S100A8 and $100A9 produce relatively more high polymers (panel E). This may be due to the too high concentration of the proteins, which increases the probability of high polymerization. Additionally, proteins themselves have aggregation-prone characteristics, and polymers may be the biochemical basis for their functional activities. Since the expression product from plasmid 4 is affected by the expression of mutant S100A8, only the homodimer of mutant S100A9 is present for this combination (see panel F). These results indicate that when the plasmids having high homologous expression are combined together, high polymerization occurs, and when the plasmids having low homologous expression are combined together, the occurrence of high polymerization would be reduced.

On the basis of the above findings, a new expression scheme is produced, in which S100A8 and S100A9-1 are cloned and expressed, and plasmid 5 is named for expressing an S100A8/S100A9 heterodimer.

### 1.3 Construction and Expression and Purification of Expression Vector 5 (Plasmid 5)

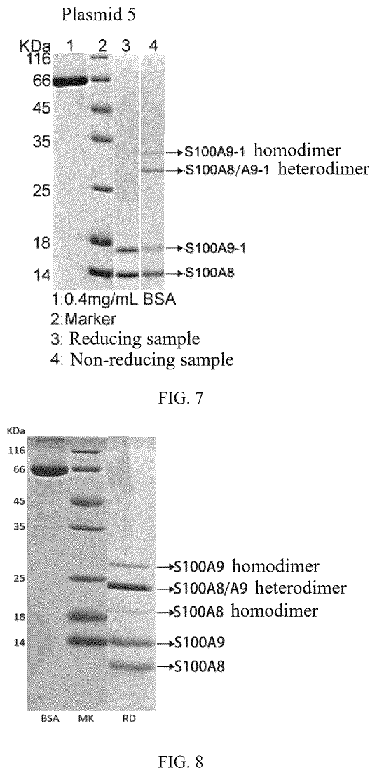

Except for the synthesized gene of interest (gene of interest S100A8+S100A9-1 in plasmid 5), the construction and expression and purification method for plasmid 5 was the same as that for plasmids 3 and 4, which was not described in detail herein. The vector map of plasmid 5 is as shown in FIG. 6, and the SDS-PAGE gel electrophoresis of the protein sample purified by His-Tag and Flag-Tag is as shown in FIG. 7.

It can be seen from the figures that an obvious band at a size of 32 KDa is present in the expression product from plasmid 5, indicating that the product contains relatively more S100A8/S100A9-1 heterodimer proteins; the detected concentration of the protein can reach 10 mg/L; and no tail band is present, showing a high purity.

In order to better verify the purity of the heterodimer, an S100A8/S100A9 heterodimer protein standard (purchased from R&D Systems, Cat. No. 8226-S8-050, purity>95%) was purchased as a control. FIG. 8 shows an SDS-PAGE electrophoretogram of the S100A8/S100A9 heterodimer protein standard (RD for short). The purity of the protein was verified by size exclusion chromatography-high performance liquid chromatography (SEC-HPLC). The specific operations comprise: a) preparing a mobile phase (50 mM PB and 500 mM NaCl, pH 6.4), performing filtration with a 0.45 μm filter membrane, and performing ultrasonic degassing for at least 10 min; b) switching to TSK superSW3000 elution column (purchased from TOSOH, Cat. No. TOSOH 18675) according to a sample to be detected; c) formulating standard solution Waters BEH200 SEC Protein Standard Mix (purchased from Waters, Cat. No. SKU: 186006518); d) loading the standard: wherein the loading volume is 20 μL, the standard concentration is 0.1 μg/μL, and buffer (200 mM PB (potassium dihydrogen phosphate and potassium dihydrogen phosphate) and 500 mM NaCl, pH 6.4) are used as a mobile phase; and e) loading the sample to be detected: wherein the loading volume is 20 μL, the protein concentration is 0.1 μg/μL, and buffer is used as a mobile phase.

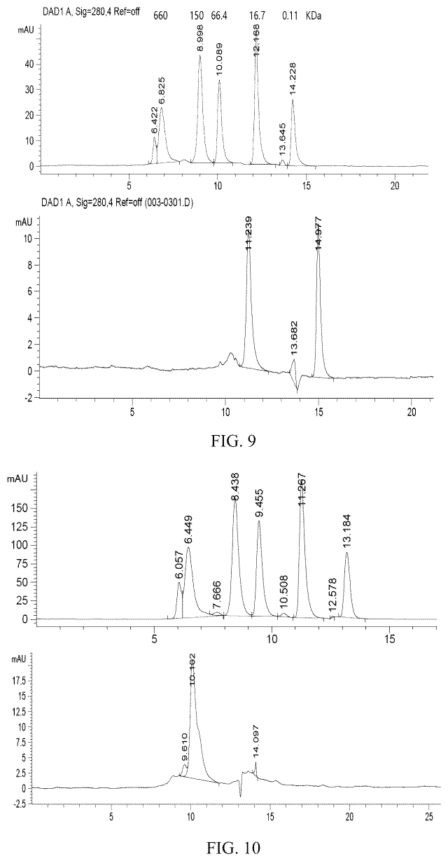

FIG. 9 shows the size exclusion chromatography-high performance liquid chromatogram of the S100A8/S100A9 heterodimer protein standard, wherein the upper panel shows the peak diagram of Maker K (standard protein molecular weight, used to guide the peak time), in which the peak times for Thyroglobulin, IgG, BSA, Myoglobin and Uracil proteins are 6.8 min, 8.9 min, 10 min, 12.1 min and 14.2 min, respectively, and the protein sizes are 660 KDa, 150 KDa, 66.4 KDa, 16.7 KDa and 11.2 KDa in turn; and the lower panel shows the peak diagram of the S100A8/S100A9 protein.

FIG. 10 shows the size exclusion chromatography-high performance liquid chromatogram of the purified S100A8/S100A9-1 recombinant protein of the present application, wherein the upper panel shows the peak diagram of Maker K, in which the peak times for Thyroglobulin, IgG, BSA, Myoglobin and Uracil proteins are 6.4 min, 8.4 min, 9.45 min, 11.27 min and 13.18 min, respectively, and the protein sizes are 660 KDa, 150 KDa, 66.4 KDa, 17 KDa and 11.2 KDa in turn; and the lower panel shows the peak diagram of the purified S100A8/S100A9-1 recombinant protein of the present application. It should be noted that due to the difference between experimental batches and between equipments, the peak time of the same protein in different experimental batches is slightly different, which is a normal phenomenon.

It can be seen from FIGS. 9-10 that the S100A8/S100A9-1 recombinant protein, the expression product from plasmid 5, was detected by SDS-PAGE and SEC-HPLC with the same main bands as the competing protein from R&D Systems, and after calculation, the S100A8/S100A9-1 recombinant protein of the present application accounts for 95% of the peak area and has a purity of more than 90%.

### 1.4 Functional Validation of S100A8/S100A9-1 Recombinant Protein

S100A8 and S100A9 can bind to TLR4 receptors and induce the NF-KB and mitogen-activated protein kinase (MAPK) pathway, leading to the secretion of pro-inflammatory cytokines (TNF, IL-6, etc.) to amplify inflammatory response. Therefore, in this example, the biological activity of S100A8/S100A9-1 recombinant protein was verified by its ability to induce human melanoma cell A375 to secrete IL-6 after treatment. Specifically, when the confluence of A375 cells reached about 80%, S100A8/S100A9-1 protein at different concentrations (0, 2.5, 5, 7.5 and 10 μg/mL) was added to the cultured A375 cells and stimulated for 48 h. The cell culture liquid was then taken. The concentration of IL-6 was detected using an ELISA kit (purchased from ABclonal, Cat. No. Human IL-6 ELISA Kit RK00004).

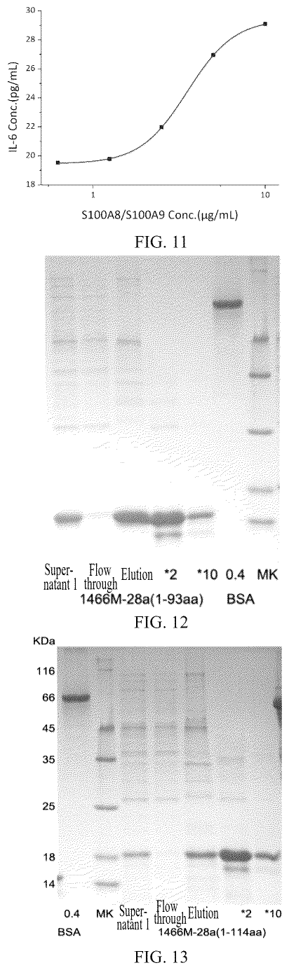

FIG. 11 shows the secretion of IL-6 after treatment with different concentrations of S100A8/S100A9-1 protein. It can be seen that the expressed and purified S100A8/S100A9-1 recombinant protein of the present application can induce cells to secrete pro-inflammatory molecule IL-6, confirming the biological activity of the recombinant protein; after calculation by fitting curve, the recombinant protein has a median effective dose (ED50) of 4.5 μg/mL for inducing secretion of protein IL-6 and has a high biological activity.

### Example 2: Preparation and Screening of Anti-S100A8 Antibody and Anti-S100A9 Antibody

### 2.1 Preparation of S100A8 and S100A9 Immunogens

(1) Preparation of S100A8 antigen: a nucleotide encoding 1-93 aa of S100A8 was synthesized by a whole gene synthesis method, and constructed into pET-28a vector for protein expression. The results were as shown in FIG. 12. The purified S100A8 recombinant protein was detected by SDS-PAGE, and had a purity of about 90% and a concentration of 1.5 mg/mL, which met the requirements for subsequent rabbit immunization and antibody screening. The antigen had an amino acid sequence as shown in SEQ ID NO: 61, and the corresponding cDNA had a nucleotide sequence as shown in SEQ ID NO: 78.

(2) Preparation of S100A9 antigen: a nucleotide encoding 1-114 aa of S100A9 was synthesized by a whole gene synthesis method, and constructed into pET-28a vector for protein expression. The results were as shown in FIG. 13. The purified S100A9 recombinant protein was detected by SDS-PAGE, and had a purity of about 90% and a concentration of 1.5 mg/mL, which met the requirements for subsequent rabbit immunization and antibody screening. The antigen had an amino acid sequence as shown in SEQ ID NO: 63, and the corresponding cDNA had a nucleotide sequence as shown in SEQ ID NO: 79.

(3) Analysis of specificity of amino acid sequences of human S100A8 protein and human S100A9 protein: By analyzing the specificity of amino acid sequences in the same species, it is convenient to find out whether the antibodies generated against these proteins may recognize other proteins. Among the S100 protein family, S100A8 and S100A9 show the highest similarity to S100A12. Sequence alignment showed that S100A8 has a 39% sequence similarity to S100A12, and S100A9 has a 47% sequence similarity to S100A12. The above alignment results indicated that S100A8 and S100A9 both have low similarity to S100A12, suggesting that the antibodies generated are expected to specifically recognize S100A8 and S100A9.

2.2 Immunization of Animals with S100A8 and S100A9 and Detection of Polyclonal Antiserum
2.2.1 Immunization of Animal with S100A8 and Detection of Polyclonal Antiserum

Four rabbits were immunized with the S100A8 recombinant protein. After the completion of the immunization program, the antiserum was taken for titer detection by ELISA and detection of clinical samples by Dot Blot. Rabbits showing the best immune response were selected for subsequent screening of rabbit monoclonal antibodies.

(1) Rabbit immunization method: Japanese white rabbits aged 3 months and weighing 2 kg were bred and housed in standard animal facilities and immunized by subcutaneous injection on the back at multiple points and intramuscular injection.

### (2) Detection of Antiserum by ELISA

The ELISA reaction conditions were as follows:
ELISA screening antigen: S100A8 (pET-28a-S100A8 (1-93 aa) expressed protein) coating concentration: 1 μg/mL, 100 μL/well
second antibody: HRP Goat Anti-Rabbit IgG (H+L), second antibody dilution ratio: 1:10000.

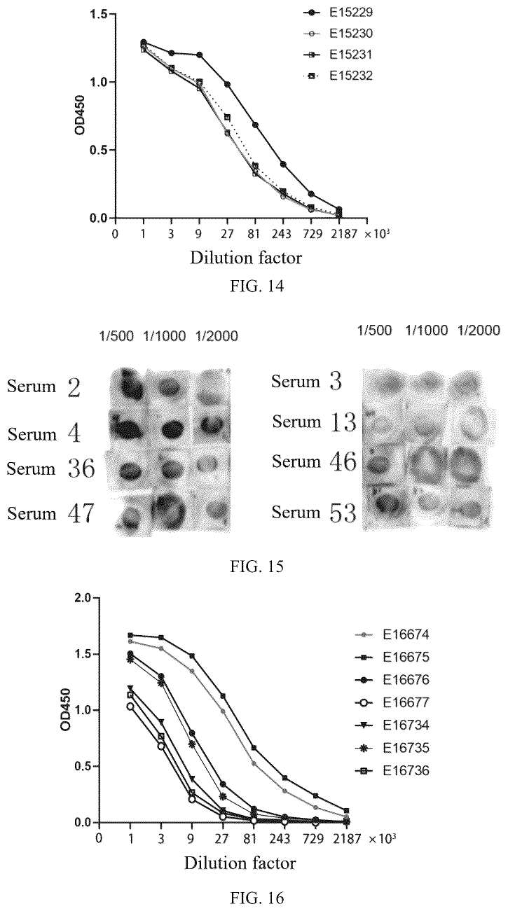

The ELISA detection results of the antiserum after the immunization with S100A8 antigen were as shown in FIG. 14. The antiserum numbered E15229 had the highest titer, which was used for the subsequent detection of clinical serum samples by Dot Blot and rabbit monoclonal antibody screening.

### (3) Detection of Clinical Serum Samples by Dot Blot

Using the antiserum of the booster immunized rabbit numbered E15229, patient sera with known concentrations of S100A8 protein were detected by Dolt blot. The sample doses and the protein concentrations corresponding to the numbers were as follows:
low concentration: serum 2=586 μg/mL, serum 3=616.6 pg/mL, serum 53=243 μg/mL. serum 46=311.8 pg/mL, serum 47=323.2 pg/mL;
medium concentration: serum 36=2924 μg/mL, and serum 13=2407.8 pg/mL;
high concentration: serum 4=11596.7 pg/mL;
the antiserum was diluted at 1/500, 1/1000 and 1/2000 respectively

The detection results were as shown in FIG. 15. A very strong S100A8 signal was detected in the patient serum 4, an S100A8 signal was detected in the patient serum 36, and relatively weak S100A8 signals were detected in the patient sera 47 and 2, which were consistent with the actual conditions of patients as serum donors. It was confirmed that the booster immunized rabbit numbered E15229 could be used for screening of rabbit monoclonal antibodies against S100A8.

2.2.2 Immunization of Animal with S100A9 and Detection of Polyclonal Antiserum

Four rabbits were immunized with the S100A9 recombinant protein. After the completion of a standard 63-day immunization program, the antiserum was taken for titer detection by ELISA and detection of clinical samples by Dot Blot. Rabbits showing the best immune response were selected for subsequent screening of rabbit monoclonal antibodies.

### (1) Rabbit Immunization Method: Same as the Immunization Method for S100A8 in Example 1

### (2) Detection of Antiserum by ELISA

The ELISA reaction conditions were as follows:
ELISA screening antigen: S100A9 antigen (pET-28a-S100A9 (1-114 aa) expressed protein)
coating concentration: 1 μg/mL, 100 μL/well
second antibody: HRP Goat Anti-Rabbit IgG (H+L), second antibody dilution ratio: 1:10000

The ELISA detection results of the antiserum after 4 immunizations with S100A9 antigen were as shown in FIG. 16, indicating that the antiserum numbered E16675 had the highest titer, which was used for the subsequent screening of rabbit monoclonal antibodies.

### (3) Detection of Clinical Serum Samples by Dot Blot

Using the rabbit antiserum numbered E16675 obtained after booster immunization, patient serum samples with known concentrations of S100A9 protein were detected by Dolt blot. The protein concentration values corresponding to the patient numbers were as follows:
low concentration: patient 2=586 μg/mL, patient 3=616.6 pg/mL, patient 53=243 μg/mL, patient 46=311.8 pg/mL, patient 47=323.2 pg/mL;
medium concentration: patient 36=2924 μg/mL, and patient 13=2407.8 pg/mL;
high concentration: patient 4=11596.7 pg/mL;
the antiserum was diluted at 1/500, 1/1000 and 1/2000 respectively

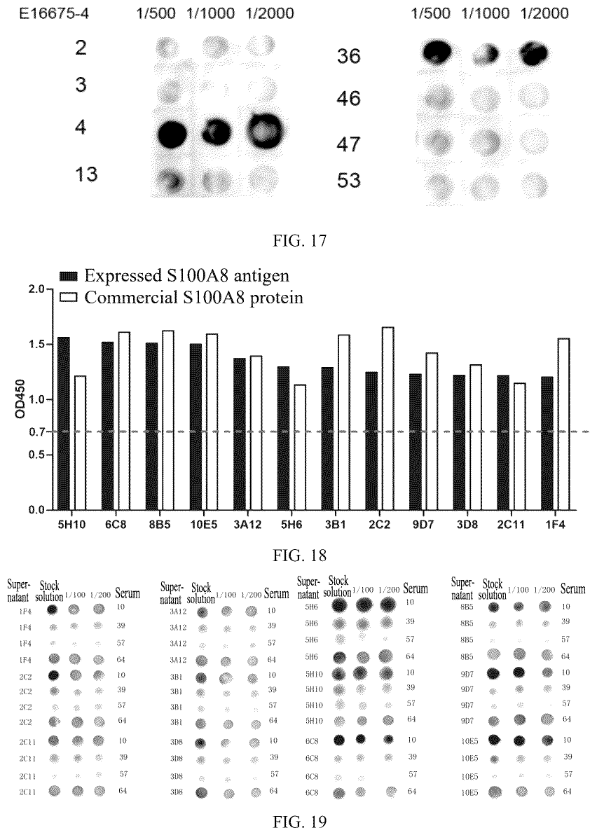

The detection results were as shown in FIG. 17. A strong S100A9 signal was detected in the serum of patient 4, relatively strong S100A9 signals were detected in the sera of patients 36 and 13, and relatively weak S100A8 signals were detected in the sera of patients 2, 3, 46, 47 and 53, which were consistent with the actual conditions of patients as serum donors, indicating that the antiserum could be used for screening of rabbit monoclonal antibodies against S100A9.

### 2.3 Screening of Rabbit Monoclonal Antibodies

### 2.3.1 Isolation of Spleen Cells and Screening of Positive B Cells

Spleen cells of rabbits after booster immunization were isolated, 1000 single B cell clones (enriched by an antigen) were sorted by a flow cytometry and cultured. Preliminary screening was performed to obtain positive clones that specifically recognized the antigen, and 50-80 μL of the supernatant from the positive clones was detected by ELISA, etc. Positive monoclones were selected for subsequent experiments.

2.3.2 Screening of Supernatant from Positive B Cells
(1) Screening of Supernatant from S100A8 Positive B Cells

A 96*10 plate was used to sort single B cells extracted from the spleen of the immunized rabbit numbered E15229. After 2 weeks of culture, the supernatants were taken for ELISA detection to screen positive clonal cell lines that can bind to the expressed S100A8 antigen. A total of 132 positive clones (with an OD value greater than 0.7) were screened. Furthermore, the binding of these 132 clones to the commercial S100A8 protein (ab167749, Recombinant human MRP8 protein) was detected by ELISA, resulting in 44 positive clones (with an OD value greater than 0.7). A total of 12 clones, namely 1F4, 2C2, 2C11, 3A12, 3B1, 3D8, 5H6, 5H10, 6C8, 8B5, 9D7 and 10E5, with the highest OD values in the two screenings were selected as candidates (FIG. 18). The supernatants from these 12 clones were used for the detection of clinical patient sera by Dot Blot. The results were as shown in FIG. 19, indicating that the signals were detected in the corresponding serum samples for all supernatants from the 12 positive B cells, which was consistent with the actual conditions of patients as serum donors.

In summary, for S100A8 antigen, a total of 12 positive B cell clones, namely 1F4, 2C2, 2C11, 3A12, 3B1, 3D8, 5H6, 5H10, 6C8, 8B5, 9D7 and 10E5, were selected for the subsequent cloning, proliferation and expression.

(2) Screening of Supernatant from S100A9 Positive B Cells

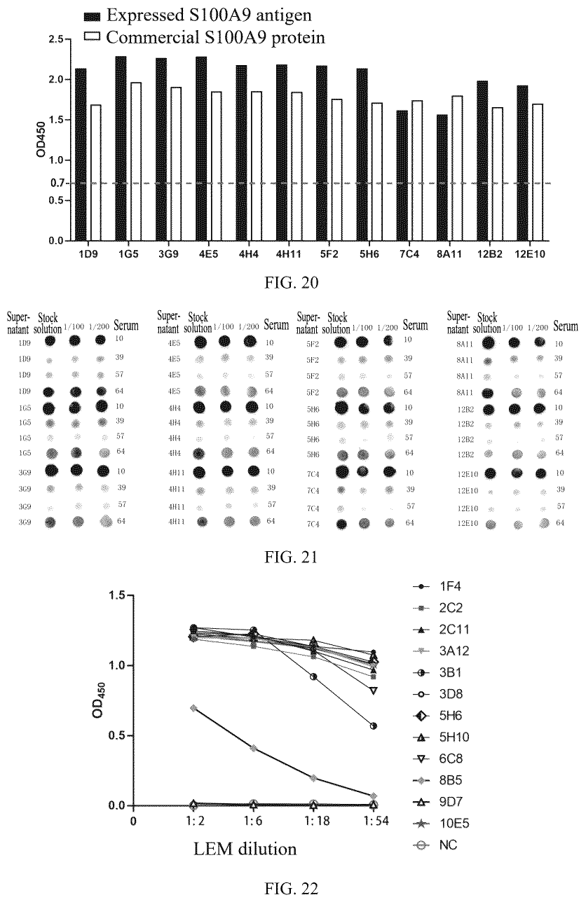

A 96*10 plate was used to sort single B cells extracted from the spleen of the immunized rabbit numbered E16675. After 2 weeks of culture, the supernatants were taken for ELISA detection to screen positive clonal cell lines that can bind to the S100A9 antigen. A total of 86 positive clones (with an OD value greater than 0.7) were screened. Furthermore, the binding of these 86 clones to the commercial S100A9 protein (ab95909, Recombinant Human S100A9 protein) was detected by ELISA, resulting in 59 positive clones. A total of 12 clones, namely 1D9, 1G5, 3G9, 4E5, 4H4, 4H11, 5F2, 5H6, 7C4, 8A11, 12B2 and 12E10, with the highest OD values in the two screenings were selected as candidates (FIG. 20). The 12 clones were used for the detection of clinical patient sera by Dot Blot. The results were as shown in FIG. 21, indicating that the signals were detected in the corresponding serum samples for all supernatants from the 12 positive B cells, which was consistent with the actual conditions of patients as serum donors.

In summary, for S100A9 antigen, a total of 12 positive B cell clones, namely 1D9, 1G5, 3G9, 4E5, 4H4, 4H11, 5F2, 5H6, 7C4, 8A11, 12B2 and 12E10, were selected for the subsequent cloning, proliferation and expression.

### 2.3.3 Proliferation and Expression of Positive Clones

The selected positive cell clones were proliferated and expressed. The antibody genes (IgG heavy chain and light chain) of a cell line of interest were cloned into an expression vector, the rabbit monoclonal antibody was recombinantly expressed in vitro, and ELISA was used to determine whether the rabbit monoclonal antibody had the antigen binding ability.

### (1) Detection of LEM (Linear Expression Module) Supernatant of S100A8

### \textcircled{1}Performing ELISA Detection and Screening Using S100A8 Recombinant Protein as a Screening Antigen

The ELISA reaction conditions were as follows:
ELISA screening antigen: S100A8 antigen (pET-28a-S100A8 (1-93 aa) expressed protein);
coating concentration: 1 μg/mL, 100 μL/well;
second antibody: HRP Goat Anti-Rabbit IgG (H+L), second antibody dilution ratio: 1:10000;

The results were as shown in FIG. 22: among the 12 LEM supernatants, except for 8B5 and 9D7, the remaining 10 LEM supernatants, namely 1F4, 2C2, 2C11, 3A12, 3B1, 3D8, 5H6, 5H10, 6C8 and 10E5, could bind to the S100A8 recombinant antigen.

\textcircled{2} performing ELISA detection and screening using commercial S100A8 protein (ab167749, Recombinant human MRP8 protein) as an antigen

The ELISA reaction conditions were as follows:
ELISA screening antigen: S100A8 antigen (commercial S100A8 protein);
coating concentration: 1 μg/mL, 100 μL/well;
second antibody: HRP Goat Anti-Rabbit IgG (H+L), second antibody dilution ratio: 1:10000;

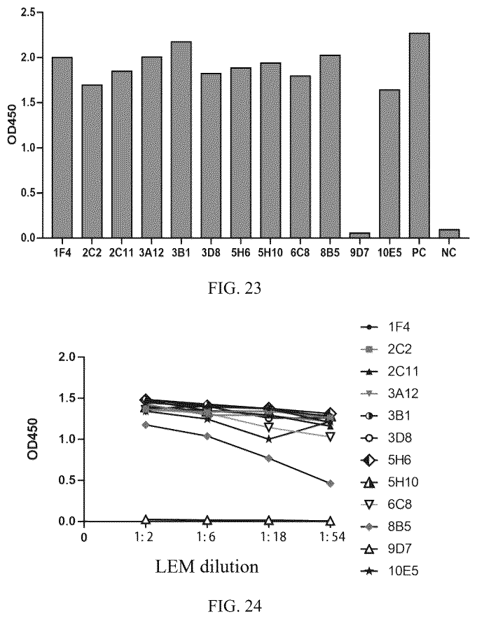

The results were as shown in FIG. 23: among the 12 LEM supernatants, except for 9D7, the 11 LEM supernatants, namely 1F4, 2C2, 2C11, 3A12, 3B1, 3D8, 5H6, 5H10, 6C8, 10E5 and 8B5, could well bind to the commercial S100A8 antigen.

\textcircled{3} performing ELISA detection and screening using S100A8/S100A9 heterodimer protein

The ELISA reaction conditions were as follows:
ELISA screening antigen: commercial S100A8/S100A9 Heterodimer (R&D) heterodimer protein;
coating concentration: 1 μg/mL, 25 μL/well;
second antibody: HRP Goat Anti-Rabbit IgG (H+L), second antibody dilution ratio: 1:5000;
the results were as shown in FIG. 24, among the 12 LEM supernatants, except for 9D7, the 11 LEM supernatants, namely 1F4, 2C2, 2C11, 3A12, 3B1, 3D8, 5H6, 5H10, 6C8, 10E5 and 8B5, could well bind to the commercial S100A8/S100A9 heterodimer protein.

### \textcircled{4} Ranking of Affinity for S100A8/S100A9 Heterodimer Protein

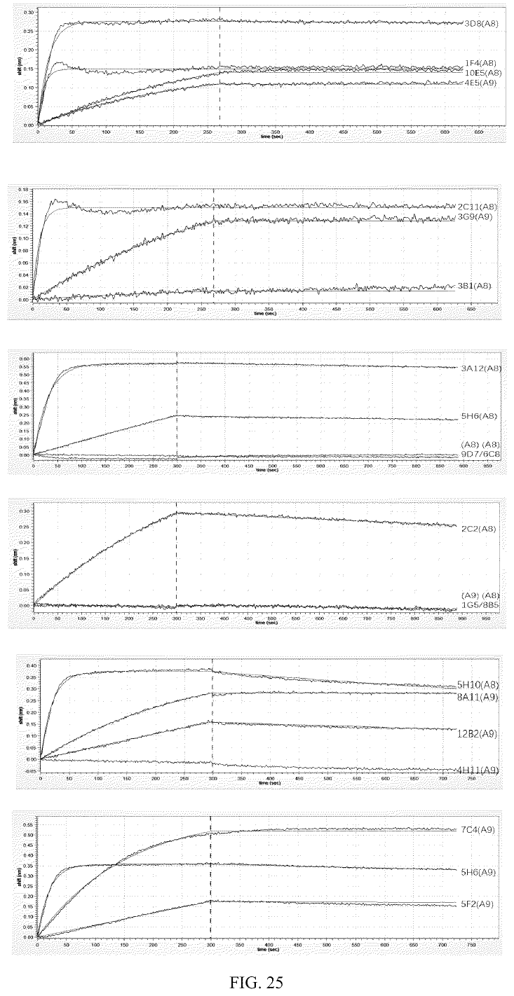

The reaction conditions used for ranking of affinity were as follows:
probe: protein A;
antibody: LEM supernatant of S100A8;
antigen: S100A8/S100A9 heterodimer protein at 100 nM;
the results were as shown in FIG. 25, according to K assay analysis, among the 12 LEM supernatants, 1F4, 2C2, 2C11, 3A12, 3D8, 5H6, 5H10 and 10E5 had relatively better capture function.

Based on the above results, a total of 3 positive clones, namely 2C2, 5H10 and 3D8, were finally selected for the expression of the recombinant monoclonal antibodies against S100A8.

### (2) Detection of LEM Supernatant of S100A9

### \textcircled{1} Performing ELISA Detection and Screening Using the Expressed S100A9 Recombinant Protein as a Screening Antigen

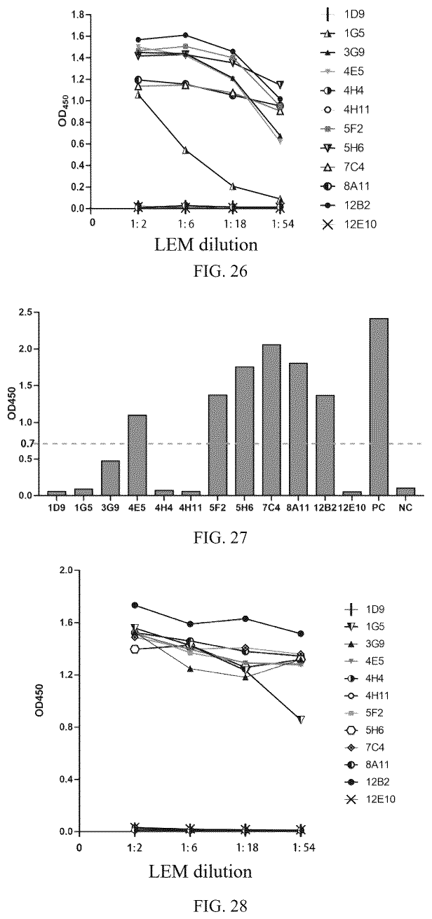

The ELISA reaction conditions were as follows:
ELISA screening antigen: S100A9 antigen (pET-28a-S100A9 (1-114 aa) expressed protein);
coating concentration: 1 μg/mL, 100 μL/well;
second antibody: HRP Goat Anti-Rabbit IgG (H+L), second antibody dilution ratio: 1:10000;
the results were as shown in FIG. 26, among the 12 LEM supernatants, the 8 LEM supernatants, namely 1G5, 3G9, 4E5, 5F2, 5H6, 7C4, 8A11 and 12B2, could well bind to the S100A9 recombinant protein.

### \textcircled{2} Performing ELISA Detection Using Commercial A9 Protein (Ab95909, Recombinant Human S100A9 Protein) as a Screening Antigen

The ELISA reaction conditions were as follows:
ELISA screening antigen: S100A9 antigen (commercial S100A9 protein);
coating concentration: 1 μg/mL, 100 μL/well;
second antibody: HRP Goat Anti-Rabbit IgG (H+L), second antibody dilution ratio: 1:10000;
the results were as shown in FIG. 27, among the 12 LEM supernatants, the 6 LEM supernatants, namely 4E5, 5F2, 5H6, 7C4, 8A11 and 12B2, could well bind to the commercial S100A9 protein.

### \textcircled{3} Performing ELISA Detection Using an S100A8/S100A9 Heterodimer Protein as a Screening Antigen

The ELISA reaction conditions were as follows:
ELISA screening antigen: S100A8/S100A9 Heterodimer (R&D) heterodimer protein;
coating concentration: 1 μg/mL, 25 μL/well;
second antibody: HRP Goat Anti-Rabbit IgG (H+L), second antibody dilution ratio: 1:5000;
the results were as shown in FIG. 28, among the 12 LEM supernatants, the 8 LEM supernatants, namely 1G5, 3G9, 4E5, 5F2, 5H6, 7C4, 8A11 and 12B2, could well bind to the S100A8/S100A9 heterodimer protein.
   \textcircled{4} ranking of affinity for S100A8/S100A9 heterodimer protein

The reaction conditions used for ranking of affinity were as follows:
probe: protein A;
antibody: LEM supernatant of S100A9;
antigen: S100A8/S100A9 heterodimer protein at 100 nM;

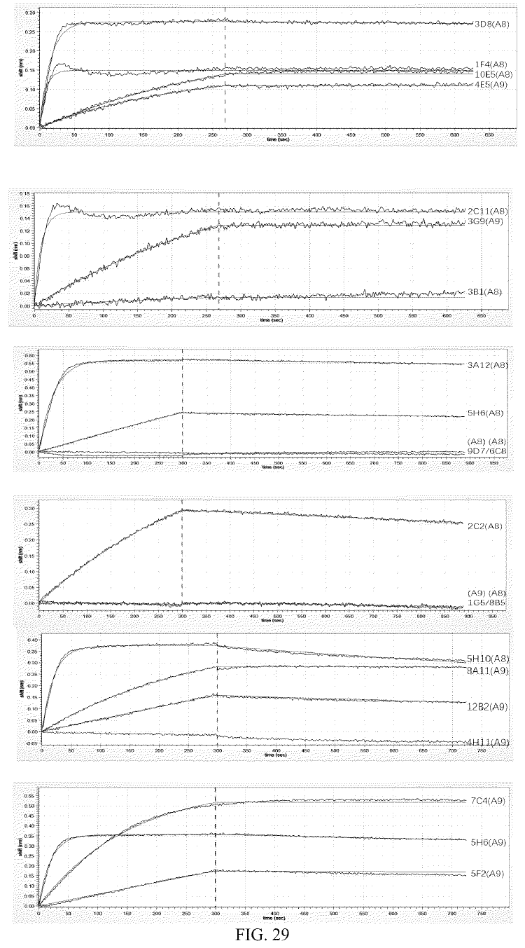

As shown in FIG. 29, according to K assay analysis, among the 12 LEM supernatants, the 7 clones, namely 3G9, 4E5, 5F2, 5H6, 7C4, 8A11 and 12B2, had relatively better capture function.

Based on the above results, a total of 2 positive clones, namely 7C4 and 8A11, were finally selected for the expression of the recombinant monoclonal antibodies against S100A9.

### 2.4 Expression of Recombinant Antibodies in a Large Quantity

The expression plasmids for the selected rabbit monoclonal antibody clones were constructed, and the recombinant rabbit monoclonal antibodies were expressed in 293F or CHO cells, and purified. The purified rabbit monoclonal antibody of at least 100 μg/clone was provided for subsequent testing. The information of the sequences of selected monoclonal antibodies was as shown in Table 5.

TABLE 5
Monoclonal antibody related sequences

| Antibody | | | |
|---|---|---|---|
| name | Name | Type | Sequence |
| 5H10 | Heavy | Amino | METGLRWLLLVAVLKGVQCQSVEESGGRLVTPGTP |
| | chain | acid | LTLTCTVSGIDLSTNPMGWVRQAPGKGLEWIGIIFA |
| | | | DTGEIYYATWARGRFTISETSSTTVDLKITSPTAEDT |
| | | | ATYFCVRGSPSSANIWGPGTLVTVSSGQPKAPSVFPL |
| | | | APCCGDTPSSTVTLGCLVKGYLPEPVTVTWNSGTLT |
| | | | NGVRTFPSVRQSSGLYSLSSVVSVTSSSQPVTCNVAH |
| | | | PATNTKVDKTVAPSTCSKPMCPPPELPGGPSVFIFPPK |
| | | | PKDTLMISRTPEVTCVVVDVSQDDPEVQFTWYINNE |
| | | | QVRTARPPLREQQFNSTIRVVSTLPIAHQDWLRGKEF |
| | | | KCKVHNKALPAPIEKTISKARGQPLEPKVYTMGPPRE |
| | | | ELSSRSVSLTCMINGFYPSDISVEWEKNGKAEDNYKT |
| | | | TPTVLDSDGSYFLYSKLSVPTSEWQRGDVFTCSVMH |
| | | | EALHNHYTQKSISRSPGK (SEQ ID NO: 41) |
| | | DNA | SEQ ID NO: 51 |
| | Light | Amino | MDTRAPTQLLGLLLLWLPGATFAAVLTQTPSPVSAA |
| | chain | acid | VGGTVTISCQSSQSVYNNNWLGWYQQKPGQPPKLLI |
| | | | YRASTLASGVSSRFKGSGSGTQFTLTISGLECDDAAT |
| | | | YYCAGTYRRNSDNGFGGGTEVVVKGDPVAPTVLIF |
| | | | PPAADQVATGTVTIVCVANKYFPDVTVTWEVDGTT |
| | | | QTTGIENSKTPQNSADCTYNLSSTLTLTSTQYNSHKE |
| | | | YTCKVTQGTTSVVQSFNRGDC |
| | | | (SEQ ID NO: 42) |
| | | DNA | SEQ ID NO: 52 |
| 2C2 | Heavy | Amino | METGLRWLLLVAVLKGVQCQSVEESGGRLVTPGGS |
| | chain | acid | LTLTCTVSGFSLSSYFMIWVRQAPGKGLEYIGIHSDT |
| | | | GSTDYASWAKGRFTISKTSTTVDLKMTSLTASDTAT |
| | | | YFCARDGGNDAYYYFNIWGPGTLVTVSSGQPKAPS |
| | | | VFPLAPCCGDTPSSTVTLGCLVKGYLPEPVTVTWNS |
| | | | GTLTNGVRTFPSVRQSSGLYSLSSVVSVTSSSQPVTC |
| | | | NVAHPATNTKVDKTVAPSTCSKPMCPPPELPGGPSV |
| | | | FIFPPKPKDTLMISRTPEVTCVVVDVSQDDPEVQFTW |
| | | | YINNEQVRTARPPLREQQFNSTIRVVSTLPIAHQDWL |
| | | | RGKEFKCKVHNKALPAPIEKTISKARGQPLEPKVYT |
| | | | MGPPREELSSRSVSLTCMINGFYPSDISVEWEKNGKA |
| | | | EDNYKTTPTVLDSDGSYFLYSKLSVPTSEWQRGDVF |
| | | | TCSVMHEALHNHYTQKSISRSPGK (SEQ ID NO: 43) |
| | | DNA | SEQ ID NO: 53 |
| | Light | Amino | MDTRAPTQLLGLLLLWLPGARCAYDMTQTPASVEV |
| | chain | acid | AVGGTVTINCQASQSISTALAWYQQKPGQPPKPLIY |
| | | | EASKLASGVPSRFKGSGSGTEFILTISDLECADAATY |
| | | | YCQQGASARNIASNVFGGGTEVVVKGDPVAPTVLIF |
| | | | PPAADQVATGTVTIVCVANKYFPDVTVTWEVDGTT |
| | | | QTTGIENSKTPQNSADCTYNLSSTLTLTSTQYNSHKE |
| | | | YTCKVTQGTTSVVQSFNRGDC (SEQ ID NO: 44) |
| | | DNA | SEQ ID NO: 54 |
| 3D8 | Heavy | Amino | METGLRWLLLVAVLKGVQCQSVEESGGRLVTPGTP |
| | chain | acid | LTLTCTVSGIDLSTNPMGWVRQAPGKGLEWIGIIFA |
| | | | DTGEIYYATWAKGRFTISETSSTTVDLKITSPTAEDT |
| | | | ATYFCVRGSPSSANIWGPGTLVTVSSGQPKAPSVFPL |
| | | | APCCGDTPSSTVTLGCLVKGYLPEPVTVTWNSGTLT |
| | | | NGVRTFPSVRQSSGLYSLSSVVSVTSSSQPVTCNVAH |
| | | | PATNTKVDKTVAPSTCSKPMCPPPELPGGPSVFIFPPK |
| | | | PKDTLMISRTPEVTCVVVDVSQDDPEVQFTWYINNE |
| | | | QVRTARPPLREQQFNSTIRVVSTLPIAHQDWLRGKEF |
| | | | KCKVHNKALPAPIEKTISKARGQPLEPKVYTMGPPRE |
| | | | ELSSRSVSLTCMINGFYPSDISVEWEKNGKAEDNYKT |
| | | | TPTVLDSDGSYFLYSKLSVPTSEWQRGDVFTCSVMH |
| | | | EALHNHYTQKSISRSPGK (SEQ ID NO: 45) |
| | | DNA | SEQ ID NO: 55 |
| | Light | Amino | MDTRAPTQLLGLLLLWLPGATFAAVLTQTPSPVSAA |
| | chain | acid | VGGTVTISCQSSQSVYNNNWLGWYQQKPGQPPKLLI |
| | | | YRASTLASGVPSRFKGSGSGTQFTFTISDLECDDAAT |
| | | | YYCAGTYRRNSDNGFGGGTEVVVKGDPVAPTVLIF |
| | | | PPAADQVATGTVTIVCVANKYFPDVTVTWEVDGTT |
| | | | QTTGIENSKTPQNSADCTYNLSSTLTLTSTQYNSHKE |
| | | | YTCKVTQGTTSVVQSFNRGDC (SEQ ID NO: 46) |
| | | DNA | SEQ ID NO: 56 |
| 7C4 | Heavy | Amino | METGLRWLLLVAVLKGVQCQSVEESGGRLVTPGTP |
| | chain | acid | LTLTCTVSGIDLSSGWMNWVRQAPGGGLEWIGTINS |
| | | | GGSPYYARWAKGRFTLSTTSTTVDLKITSPTTEDTAT |
| | | | YFCGSRDFWGPGTLVTVSSGQPKAPSVFPLAPCCGD |
| | | | TPSSTVTLGCLVKGYLPEPVTVTWNSGTLTNGVRTFP |
| | | | SVRQSSGLYSLSSVVSVTSSSQPVTCNVAHPATNTKV |
| | | | DKTVAPSTCSKPMCPPPELPGGPSVFIFPPKPKDTLMI |
| | | | SRTPEVTCVVVDVSQDDPEVQFTWYINNEQVRTARP |
| | | | PLREQQFNSTIRVVSTLPIAHQDWLRGKEFKCKVHN |
| | | | KALPAPIEKTISKARGQPLEPKVYTMGPPREELSSRSV |
| | | | SLTCMINGFYPSDISVEWEKNGKAEDNYKTTPTVLD |
| | | | SDGSYFLYSKLSVPTSEWQRGDVFTCSVMHEALHNH |
| | | | YTQKSISRSPGK (SEQ ID NO: 47) |
| | | DNA | SEQ ID NO: 57 |
| | Light | Amino | MDTRAPTQLLGLLLLWLPGATFAQVLTQTASPVSAA |
| | chain | acid | VGGTVTINCQASQSVYNNNYLAWYQQKPGQPPKRL |
| | | | IYAASNLASGVSSRFKGSGSGTQFTLTISDLQCDDAA |
| | | | TYYCSGGYDCDSADCSVFGGGTEVVVKGDPVAPTV |
| | | | LIFPPAADQVATGTVTIVCVANKYFPDVTVTWEVDG |
| | | | TTQTTGIENSKTPQNSADCTYNLSSTLTLTSTQYNSH |
| | | | KEYTCKVTQGTTSVVQSFNRGDC (SEQ ID NO: 48) |
| | | DNA | SEQ ID NO: 58 |
| 8A11 | Heavy | Amino | METGLRWLLLVAVLKGVQCQSVEESGGRLVTPGTP |
| | chain | acid | LTLTCTVSGIDLSSGWMNWVRQAPGKGLEWIGVIN |
| | | | SYGRTYYARWAKGRFTISITSTTVDLKITSPTTEDTA |
| | | | TYFCGSRDFWGPGTLVTVSSGQPKAPSVFPLAPCCG |
| | | | DTPSSTVTLGCLVKGYLPEPVTVTWNSGTLINGVRT |
| | | | FPSVRQSSGLYSLSSVVSVTSSSQPVTCNVAHPATNT |
| | | | KVDKTVAPSTCSKPMCPPPELPGGPSVFIFPPKPKDTL |
| | | | MISRTPEVTCVVVDVSQDDPEVQFTWYINNEQVRTA |
| | | | RPPLREQQFNSTIRVVSTLPIAHQDWLRGKEFKCKVH |
| | | | NKALPAPIEKTISKARGQPLEPKVYTMGPPREELSSRS |
| | | | VSLTCMINGFYPSDISVEWEKNGKAEDNYKTTPTVL |
| | | | DSDGSYFLYSKLSVPTSEWQRGDVFTCSVMHEALHN |
| | | | HYTQKSISRSPGK (SEQ ID NO: 49) |
| | | DNA | SEQ ID NO: 59 |
| | Light | Amino | MDTRAPTQLLGLLLLWLPGATFAQVLTQTASPVSAA |
| | chain | acid | VGSTVTINCQASQSVYKNNYLAWYQQKPGQPPKRLI |
| | | | YSASTLASGVSSRFKGSGSGTQFTLTISDVQCDDAAT |
| | | | YYCGGGYDCDSADCSVFGGGTEVVVKGDPVAPTV |
| | | | LIFPPAADQVATGTVTIVCVANKYFPDVTVTWEVDG |
| | | | TTQTTGIENSKTPQNSADCTYNLSSTLTLTSTQYNSH |
| | | | KEYTCKVTQGTTSVVQSFNRGDC (SEQ ID NO: 50) |
| | | DNA | SEQ ID NO: 60 |

Note: in the above-mentioned amino acid sequences of the heavy chain/light chain, the signal peptide sequence is located upstream of the sequence and marked by the underlined single line; the constant region sequence is located downstream of the sequence and marked by the underlined double line; and the complementarity-determining region (CDR) sequence is located in the middle of the sequence and displayed in the bold font.

### Example 3: Test of Pairing Between Monoclonal Antibody Against S100A8 and Monoclonal Antibody Against S100A9

Three anti-S100A8 antibodies (5H10, 2C2 and 3D8) and two anti-S100A9 antibodies (7C4 and 8A11) which are confirmed in the previous screening were paired with each other.

Specific experiment procedure:
coating with a coating antibody: coating (384-well plate) with the coating antibody at 4° C. overnight;
coating antibody concentration: 2 μg/mL, 25 μL/well;
blocking solution: 50 L/well, incubating at room temperature for 1 h;
protein: S100A8/S100A9 Heterodimer (R&D), 3-fold gradient dilution from 1 μg/mL, with a total of 7 concentrations, or 2-fold gradient dilution from 40 μg/mL, with a total of 7 concentrations, 25 μL/well, incubating at room temperature for 1.5 h
detecting antibody: detecting antibody (biotin-labeled)
detecting antibody concentration: 2 μg/mL, 25 μL/well, incubating at room temperature for 1 h;
HRP: Neutravidin-HRP, dilution ratio of 1:10000, 25 μL/well, incubating at room temperature for 1 h;
color development: TMB [Moss INS, TWBHK-1000] color development (1:5), 25 μL/well, performing color development at room temperature for 10 min in the dark.

The results were as follows:

### 3.1 the Coating Antibody is Anti-S100A8 Antibody, and the Detecting Antibody is Anti-S100A9 Antibody (Biotin-Labeled):

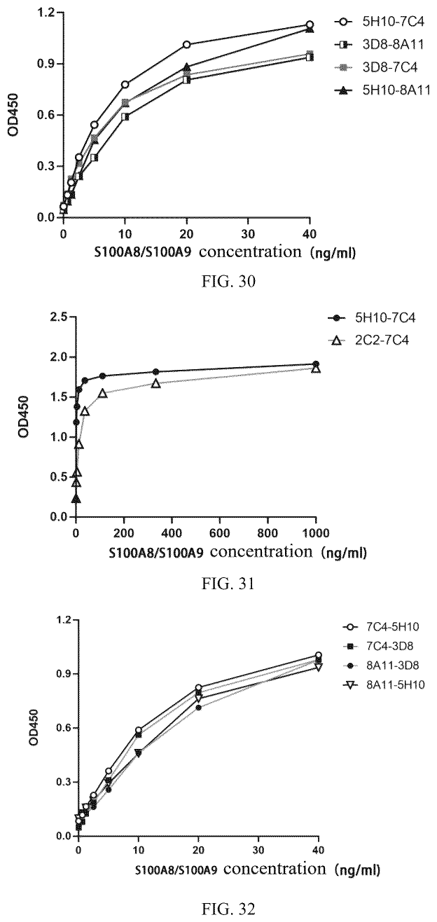

As shown in FIG. 30 and FIG. 31, the five antibody pairs, namely 5H10-7C4 (biotin-labeled), 5H10-8A11 (biotin-labeled), 3D8-7C4 (biotin-labeled), 3D8-8A11 (biotin-labeled) and 2C2-7C4 (biotin-labeled), showed good pairing. Among them, the antibody pair 5H10-7C4 had the best detection effect.

### 3.2 the Coating Antibody is Anti-S100A9 Antibody, and the Detecting Antibody is Anti-S100A8 Antibody (Biotin-Labeled):

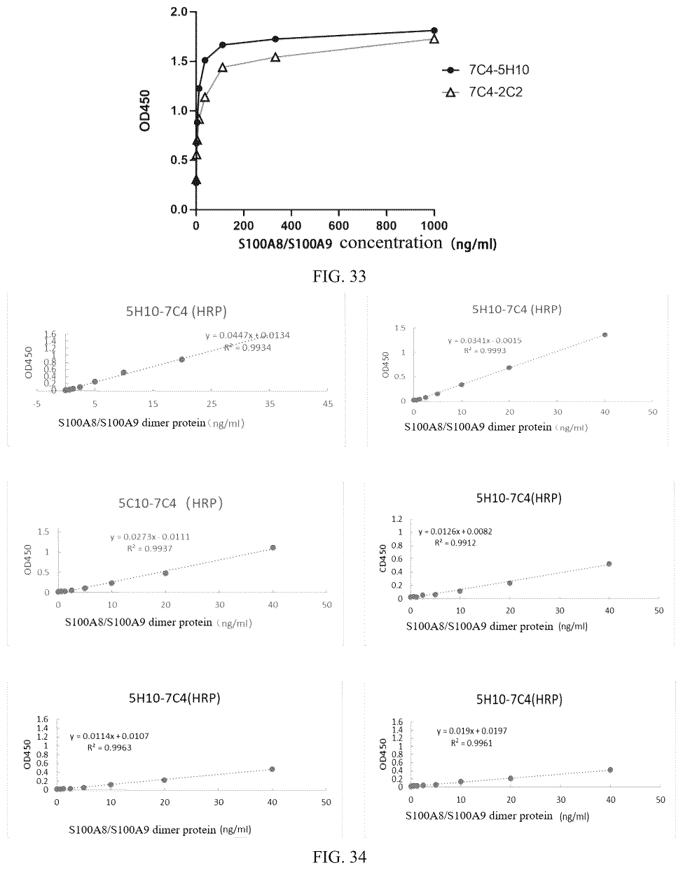

As shown in FIG. 32 and FIG. 33, the five antibody pairs, namely 7C4-5H10 (biotin-labeled), 7C4-3D8 (biotin-labeled), 8A11-5H10 (biotin-labeled), 8A11-3D8 (biotin-labeled) and 7C4-2C2 (biotin-labeled), showed good pairing. Among them, the antibody pair 7C4-5H10 (biotin-labeled) had the best detection effect.

In summary, all the five antibody pairs showed good detection of S100A8/S100A9 protein, among which the antibody pair 5H10-7C4 had the best detection effect. Furthermore, the result of the antibody pair in which the coating antibody was an anti-S100A8 antibody and the detecting antibody was an anti-S100A9 antibody was better than the result of the antibody pair in which the coating antibody was an anti-S100A9 antibody and the detecting antibody was an anti-S100A8 antibody. The antibody pair 5H10-7C4 (biotin-labeled) was finally selected for subsequent detection of patient sera.

### Example 4: Detection of Clinical Samples Using Antibody Pair of Anti-S100A8 Antibody and Anti-S100A9 Antibody

### 4.1 Information of Enrolled Patients

The baseline information of healthy individuals and patients who were used for detection using antibody pair 5H10-7C4 (the detecting antibody 7C4 was directly labeled with HRP) was shown in Table 6 below.

TABLE 6
Baseline information of healthy individuals and patients

| | | | Patients with coronary | | |
|---|---|---|---|---|---|
| | Healthy individuals | | heart disease | | P |
| Variable | (n = 20) | | (n = 15) | | value |
| Gender (male/ | 3/17 | | 14/1 | | <0.001 |
| female) | | | | | |
| Age/years | 52.5 | (46.8, 59.3) | 37.5 | (33.3, 40.0) | <0.001 |
| SBP/(mmHg) | 120.0 | (107.3, 131.5) | 122.5 | (120.0, 133.5) | 0.236 |
| DBP/(mmHg) | 78.0 | (69.3, 85.5) | 80.0 | (70.8, 83.0) | 0.369 |
| Smoking | 3 | (15.0) | 15 | (29.4) | 0.091 |
| history/(%) | | | | | |
| Blood glucose/ | 5.6 | (5.2, 5.9) | 5.1 | (4.8, 6.1) | 0.409 |
| (mmol/L) | | | | | |
| Cholesterol/ | 2.3 | (1.3, 5.1) | 4.4 | (3.5, 5.7) | 0.024 |
| (mmol/L) | | | | | |
| LDL/(mmol/L) | 1.8 | (1.4, 3.4) | 2.7 | (2.1, 3.4) | 0.158 |

### 4.2 Collection and Processing Steps of Patient Serum Samples

Patient blood samples were collected in strict accordance with the standard procedures upon admission, and were immediately centrifuged at room temperature (2400 g, 10 min) to isolate serum. After centrifugation, the serum samples were subpackaged in equal portions, labeled with corresponding numbers. The subpackaged samples were placed in a−80° C. refrigerator for cryopreservation.

Blood samples of healthy population were obtained from the physical examination center and were also collected and preserved in accordance with the same procedures mentioned above.

### 4.3 Detection of S100A8/S100A9 Level in Clinical Samples Using Commercial ELISA Kit

Experimental steps:
Kit: R&D Systems, Inc, Human S100A8/S100A9, Heterodimer Immunoassay
(1) Preparation of reagents:

1) all reagents were equilibrated to room temperature before use.

2) Wash Buffer: if a crystal was formed in a concentrated liquor, the concentrate liquor was equilibrated to room temperature and gentle shaking was performed until the crystal was completely dissolved, ionized water or distilled water was added to dilute 20 mL of Wash Buffer to a total volume of 500 mL.

3) Substrate solution: chromogenic reagents A and B should be mixed at equal volumes 15 min before use and preserved in the dark, wherein 200 μL of the mixture comprising equal volumes of chromogenic reagents A and B was added to each well.

4) S100A8/S100A9 standard: the S100A8/9 standard was reprepared using calibrator diluent RD5-10. The reprepared product was a 40 ng/mL stock solution. The standard was stirred gently for at least 15 min before dilution. 250 μL of the appropriate calibrator diluent RD5-10 was pipetted and transferred to each tube. A series of diluents (20 ng/ml, 10 ng/ml, 5 ng/ml, 2.5 ng/ml, 1.25 ng/mL, 0.625 ng/mL) were prepared using the stock solution, with the undiluted standard (40 ng/mL) as the high concentration standard and the standard diluent as the zero standard (0 pg/mL).

(2) Determination steps:
all reagents and samples were equilibrated to room temperature before use, and all samples, standards and controls were determined in duplicate.

1) preparing all reagents and working standards, and diluting serum samples 150 times;

2) removing the excess microplate strips, putting same back into a tin foil bag with a desiccant, and re-sealing the bag;

3) adding 50 μL of Assay Diluent RD1-34 to each well;

4) sequentially adding 50 μL of standard, sample and control to each well, sealing the wells with rubber strips, performing incubation at room temperature for 2 h, and recording the distribution of the determination standards and samples;

5) sucking and discarding the liquid in the wells, completely washing the wells with a Wash Buffer at 400 μL/well, completely removing the buffer, tapping the plate on clean paper until dry, and washing the wells repeatedly 4 times;

6) adding 200 μL of S100A8/S100A9 Conjugate to each well, sealing the wells with new rubber strips, and performing warm bath at room temperature for 2 h;

7) repeating step 5;

8) adding 200 μL of Substrate Solution to each well, and performing incubation in the dark at room temperature for 30 min;

9) adding 50 μL of Stop Solution to each well, wherein the color in the well should change from blue to yellow, and if the color in the well is green, or the color changes unevenly, the plate is gently tapped to ensure sufficient mixing;

and 10) within 30 min, determining the absorbance of each well at 450 nm using a microplate reader, wherein if wavelength correction is available, the wavelength is set to 540 nm or 570 nm, and if wavelength correction is not available, the reading at the wavelength of 540 nm or the reading at the wavelength of 570 nm is subtracted from the reading at the wavelength of 450 nm. This method can correct the optical defects of the plate. Direct reading at 450 nm without correction may be higher or lower.

(3) Calculation of results:
1) the OD value of the zero standard was subtracted from the OD values of the standards, controls and samples, and the average value of the duplicate wells was taken.
2) using a 4-parameter curve and computer software, a standard curve was established.
3) if the sample was diluted, the concentration obtained from the standard curve must be multiplied by the dilution factor.

### 4.4 Detection of S100A8/S100A9 Level in Clinical Samples Using Antibody Pair 5H10-7C4 by ELISA

(1) Experimental steps:
standard: commercial R&D S100A8/S100A9 heterodimer protein, 2-fold gradient dilution from 40 ng/mL, with a total of 8 concentrations, lower limit of 0.625 ng, and boundary value of 40-0.625;
reaction system: 96-well plate reaction system, laboratory buffer;
coating antibody concentration: 5H10 (anti-S100A8 antibody), 0.5 μg/mL;
detecting antibody concentration: 7C4-HRP (directly labeled), antibody dilution ratio of 1:20000 or 1:10000;
TMB development: dilution ratio being 1:2, and color development time being 15 min.

(2) Linear Range and Sensitivity of Standard Curve:

The linear range and sensitivity of the standard curve for the antibody pair 5H10-7C4 (HRP) in 6 batches of experiments were as shown in FIG. 34 and Table 7.

TABLE 7
Analysis of linear range and sensitivity for
5H10-7C4 (HRP) in 6 batches of experiments

| | | | | Linear range |
|---|---|---|---|---|
| | Linear | | | after calibration |
| | range | Sensitivity | | with serum |
| Antibody pair | (ng/mL) | (ng/ml) | R2 | (ng/mL) |
| 5H10-7C4 (first batch) | 0-20 | 0.625 | 0.9934 | 2.5-40 |
| 5H10-7C4 (second batch) | 0-40 | 1.25 | 0.9993 | 2.5-40 |
| 5H10-7C4 (third batch) | 0-40 | 1.25 | 0.9937 | 2.5-40 |
| 5H10-7C4 (fourth batch) | 0-20 | 2.5 | 0.9912 | 5-40 |
| 5H10-7C4 (fifth batch) | 0-40 | 5 | 0.9963 | 5-40 |
| 5H10-7C4 (sixth batch) | 0-40 | 5 | 0.9961 | 5-40 |

(3) Results of Detection of Clinical Samples with Developed Antibody Pair

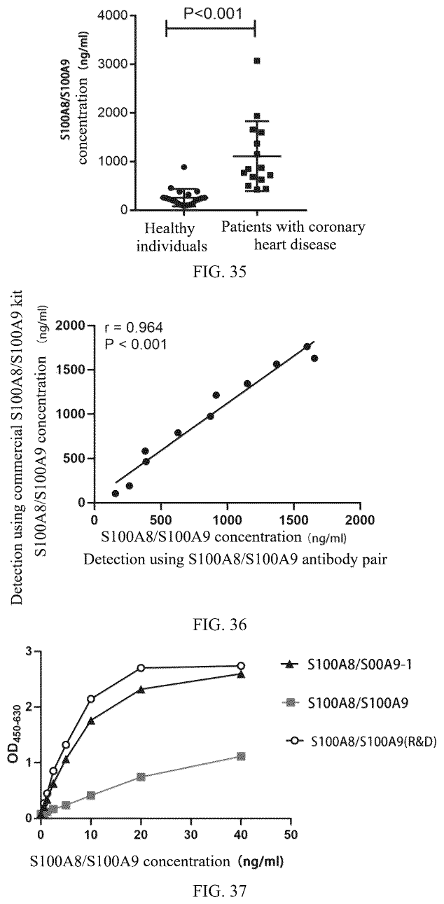

The developed 5H10-7C4 (the detecting antibody 7C4 had a HRP label) could effectively distinguish the concentration of S100A8/S100A9 in the serum of healthy individuals and patients with coronary heart disease (FIG. 35). The concentration of S100A8/S100A9 in the clinical sample serum which was detected with the developed 5H10-7C4 antibody pair was highly matched with the detection results of the commercial S100A8/S100A9 kit (FIG. 36).

### Example 5: Evaluation of S100A8/S100A9 Heterodimer Standard

### 5.1 Source of S100A8/S100A9 Heterodimer

The S100A8/S100A9 heterodimer prepared in example 1 was used, wherein S100A8 was wild type (amino acid sequence being SEQ ID NO: 61) and S100A9-1 carried E9A mutation (amino acid sequence being SEQ ID NO: 67). The purpose of constructing an S100A9 mutant was to improve the stability of the S100A8/S100A9 heterodimer without changing its epitope.

### 5.2 Detection of Binding of Purified S100A8/S100A9 Heterodimer to Antibody Pair (5H10-7C4-HRP) by ELISA

Antibody coating: coating a 96-well plate with the screened anti-S100A8 monoclonal antibody 5H10 (0.5 μg/mL) at 100 μl/well at 4° C. overnight;

Blocking: adding ELISA blocking solution at 200 μL/well, and incubating the plate at room temperature for 1 h;

Antigen: performing 3-fold gradient dilution from 40 ng/ml on the expressed and purified S100A8/S100A9 heterodimer, S100A8/S100A9-1 (S100A9 mutant) heterodimer and commercial S100A8/S100A9 protein, with a total of 7 concentrations, adding the resulting diluents to an ELISA plate at 100 μl/well, and performing incubation at room temperature for 1 h;

Detecting antibody: diluting the screened anti-S100A9 monoclonal antibody 7C4-HRP at 1:10000, adding the resulting diluents to each well at 100 μl/well, and performing incubation at room temperature for 1 h;

Color development: adding TMB [Thermo Fisher, 34029] (1:2) to each well at 100 μL/well, and performing color development in the dark at room temperature for 10 min.

The detection results were as shown in FIG. 37, the antibody pair 5H10-7C4-HRP provided by the present application could be recognized by the S100A8/S100A9 heterodimer; and the binding ability of the S100A8/S100A9-1 heterodimer provided by the present application to the antibody pair was highly matched with that of the commercial S100A8/S100A9 dimer to the antibody pair.

It should be stated that the above are only the preferred examples of the present application and are not intended to limit the present application. For those of ordinary skill in the art, various modifications and changes can be made to the present application. Although the specific embodiments have been described, for the applicant or those of ordinary skill in the art, the substitutions, modifications, changes, improvements, and substantial equivalents of the above embodiments may exist or cannot be foreseen currently. Therefore, the submitted appended claims and claims that may be modified are intended to cover all such substitutions, modifications, changes, improvements, and substantial equivalents. It is important that, as the technology evolves, many elements described herein may be replaced with equivalent elements that appear after the present application.

1. An antibody or an antigen-binding fragment thereof that binds to S100A8 or S100A9, wherein the antibody or the antigen-binding fragment at least comprises one group or more groups selected from groups (i)-(iii) and/or groups (iv)-(v):
(i) sequences as shown in SEQ ID NOs: 1, 2, 3, 4 and 6, and amino acid sequence RAS;
(ii) sequences as shown in SEQ ID NOs: 7, 8, 9, 10 and 12, and amino acid sequence EAS;
(iii) sequences as shown in SEQ ID NOs: 13, 14, 15, 16 and 18, and amino acid sequence RAS;
(iv) sequences as shown in SEQ ID NOs: 19, 20, 21, 22 and 24, and amino acid sequence AAS; and/or
(v) sequences as shown in SEQ ID NOs: 25, 26, 27, 28 and 30, and amino acid sequence SAS.

2. The antibody or the antigen-binding fragment thereof according to claim 1, comprising one or more group of amino acid sequences selected from groups (i)-(iii) and/or groups (iv)-(v):
(i) a heavy chain variable region (VH) having an amino acid sequence as shown in SEQ ID NO: 31 and a light chain variable region (VL) having an amino acid sequence as shown in SEQ ID NO: 32;
(ii) VH having an amino acid sequence as shown in SEQ ID NO: 33 and VL having an amino acid sequence as shown in SEQ ID NO: 34;
(iii) VH having an amino acid sequence as shown in SEQ ID NO: 35 and VL having an amino acid sequence as shown in SEQ ID NO: 36;
(iv) VH having an amino acid sequence as shown in SEQ ID NO: 37 and VL having an amino acid sequence as shown in SEQ ID NO: 38;
(v) VH having an amino acid sequence as shown in SEQ ID NO: 39 and VL having an amino acid sequence as shown in SEQ ID NO: 40; and/or
(vi) an amino acid sequence having at least 80%, 85%, 90%, 95%, 98% or 99% identity to VH and/or VL in each of the aforementioned groups.

3. A polynucleotide encoding the antibody or the antigen-binding fragment thereof according to claim 1.

4. A recombinant vector comprising the polynucleotide according to claim 3.

5. A host cell comprising the polynucleotide according to claim 3.

6. A polypeptide comprising an amino acid sequence having at least 80%, 85%, 90%, 95%, 98% or 99% identity to a sequence as shown in any one of SEQ ID NO: 61, SEQ ID NO: 65, SEQ ID NO: 63 and SEQ ID NO: 67;
and optionally wherein the polypeptide comprises a sequence as shown in any one of SEQ ID NO: 61, SEQ ID NO: 65, SEQ ID NO: 63 and SEQ ID NO: 67.

7. A heterodimer of S100A8 and S100A9, wherein the heterodimer comprises amino acid sequences having at least 80%, 85%, 90%, 95%, 98% or 99% identity to sequences as shown in SEQ ID NO: 61 and SEQ ID NO: 67;
and optionally wherein the heterodimer comprises sequences as shown in SEQ ID NO: 61 and SEQ ID NO: 67.

8. A polynucleotide, wherein the polynucleotide encodes the polypeptide according to claim 6.

9. An expression vector comprising the polynucleotide according to claim 8.

10. An expression vector encoding the heterodimer according to claim 7, wherein the expression vector comprises an S100A8 fusion gene and an S100A9 fusion gene;
wherein the S100A8 fusion gene comprises a first promoter and the S100A8 gene which are operably linked, and the S100A8 gene has a nucleic acid sequence as shown in SEQ ID NO: 62; and
wherein the S100A9 fusion gene comprises a second promoter and the S100A9 gene which are operably linked, and the S100A9 gene has a nucleic acid sequence as shown in SEQ ID NO: 68.

11. The expression vector according to claim 10, wherein the first promoter and the second promoter are the same, and are selected from one of a lac promoter, a trp promoter, a tac promoter, a 1pl promoter or a T7 promoter;
and optionally wherein the first promoter and the second promoter are T7 promoters, and the T7 promoter has a nucleic acid sequence as shown in SEQ ID NO: 69.

12. A host cell, wherein the host cell comprises the polynucleotide according to claim 8.

13. A kit comprising the antibody or the antigen-binding fragment thereof according to claim 1, the polypeptide according to claim 6, or the heterodimer according to claim 7.

14. The kit according to claim 13, wherein the kit comprises a capture antibody and a labeled antibody,
wherein the capture antibody comprises a VH and a VL selected from one or more group of groups (i)-(iii):
(i) a VH comprising an amino acid sequence as shown in SEQ ID NO: 31 and a VL comprising an amino acid sequence as shown in SEQ ID NO: 32;
(ii) a VH comprising an amino acid sequence as shown in SEQ ID NO: 33 and a VL comprising an amino acid sequence as shown in SEQ ID NO: 34; and/or
(iii) a VH comprising an amino acid sequence as shown in SEQ ID NO: 35 and a VL comprising an amino acid sequence as shown in SEQ ID NO: 36;
wherein the labeled antibody comprises a VH and a VL is selected from one group or more groups of groups (iv)-(v):
(iv) a VH comprising an amino acid sequence as shown in SEQ ID NO: 37 and a VL comprising an amino acid sequence as shown in SEQ ID NO: 38; and/or
(v) a VH comprising an amino acid sequence as shown in SEQ ID NO: 39 and a VL comprising an amino acid sequence as shown in SEQ ID NO: 40.

15. The kit according to claim 14, wherein:
the VH of the capture antibody comprises a sequence as shown in SEQ ID NO: 31, the VL of the capture antibody comprises a sequence as shown in SEQ ID NO: 32, the VH of the labeled antibody comprises a sequence as shown in SEQ ID NO: 37, and the VL of the labeled antibody comprises a sequence as shown in SEQ ID NO: 38;
the VH of the capture antibody comprises a sequence as shown in SEQ ID NO: 31, the VL of the capture antibody comprises a sequence as shown in SEQ ID NO: 32, the VH of the labeled antibody comprises a sequence as shown in SEQ ID NO: 39, and the VL of the labeled antibody comprises a sequence as shown in SEQ ID NO: 40;
the VH of the capture antibody comprises a sequence as shown in SEQ ID NO: 33, the VL of the capture antibody comprises a sequence as shown in SEQ ID NO: 34, the VH of the labeled antibody comprises a sequence as shown in SEQ ID NO: 37, and the VL of the labeled antibody comprises a sequence as shown in SEQ ID NO: 38;
the VH of the capture antibody comprises a sequence as shown in SEQ ID NO: 35, the VL of the capture antibody comprises a sequence as shown in SEQ ID NO: 36, the VH of the labeled antibody comprises a sequence as shown in SEQ ID NO: 37, and the VL of the labeled antibody comprises a sequence as shown in SEQ ID NO: 38; or
the VH of the capture antibody comprises a sequence as shown in SEQ ID NO: 35, the VL of the capture antibody comprises a sequence as shown in SEQ ID NO: 36, the VH of the labeled antibody comprises a sequence as shown in SEQ ID NO: 39, and the VL of the labeled antibody comprises a sequence as shown in SEQ ID NO: 40.

16. The kit according to claim 13, wherein the kit comprises a capture antibody and a labeled antibody,
wherein the labeled antibody comprises a VH and a VL selected from one group or more groups of groups (i)-(iii):
(i) a VH comprising an amino acid sequence as shown in SEQ ID NO: 31 and a VL comprising an amino acid sequence as shown in SEQ ID NO: 32;
(ii) a VH comprising an amino acid sequence as shown in SEQ ID NO: 33 and a VL comprising an amino acid sequence as shown in SEQ ID NO: 34; and/or
(iii) a VH comprising an amino acid sequence as shown in SEQ ID NO: 35 and a VL comprising an amino acid sequence as shown in SEQ ID NO: 36;
wherein the capture antibody is selected from one group or more groups of groups (iv)-(v):
(iv) a VH comprising an amino acid sequence as shown in SEQ ID NO: 37 and a VL comprising an amino acid sequence as shown in SEQ ID NO: 38; and/or
(v) a VH comprising an amino acid sequence as shown in SEQ ID NO: 39 and a VL comprising an amino acid sequence as shown in SEQ ID NO: 40.

17. The kit according to claim 16, wherein:
the VH of the capture antibody comprises a sequence as shown in SEQ ID NO: 37, the VL of the capture antibody comprises a sequence as shown in SEQ ID NO: 38, the VH of the labeled antibody comprises a sequence as shown in SEQ ID NO: 31, and the VL of the labeled antibody comprises a sequence as shown in SEQ ID NO: 32;
the VH of the capture antibody comprises a sequence as shown in SEQ ID NO: 37, the VL of the capture antibody comprises a sequence as shown in SEQ ID NO: 38, the VH of the labeled antibody comprises a sequence as shown in SEQ ID NO: 35, and the VL of the labeled antibody comprises a sequence as shown in SEQ ID NO: 36;
the VH of the capture antibody comprises a sequence as shown in SEQ ID NO: 39, the VL of the capture antibody comprises a sequence as shown in SEQ ID NO: 40, the VH of the labeled antibody comprises a sequence as shown in SEQ ID NO: 31, and the VL of the labeled antibody comprises a sequence as shown in SEQ ID NO: 32;
the VH of the capture antibody comprises a sequence as shown in SEQ ID NO: 39, the VL of the capture antibody comprises a sequence as shown in SEQ ID NO: 40, the VH of the labeled antibody comprises a sequence as shown in SEQ ID NO: 35, and the VL of the labeled antibody comprises a sequence as shown in SEQ ID NO: 36; or
the VH of the capture antibody comprises a sequence as shown in SEQ ID NO: 37, the VL of the capture antibody comprises a sequence as shown in SEQ ID NO: 38, the VH of the labeled antibody comprises a sequence as shown in SEQ ID NO: 33, and the VL of the labeled antibody comprises a sequence as shown in SEQ ID NO: 34.

18. The kit according to claim 14 or 16, wherein the labeled antibody comprises a detectable label, wherein the detectable label comprises an enzyme, a chemiluminescent reagent, a fluorescent dye, colloidal gold or biotin, and optionally wherein the detectable label is biotin.

19. The kit according to claim 13, wherein the kit is used to detect S100A8, S100A9, and/or a dimer of S100A8 and S100A9 in a sample by immunoassay;
optionally wherein the immunoassay comprises an enzyme linked immunosorbent assay (ELISA) method, an immunoblotting method, an immunohistochemistry method, an immunofluorescence method, a radioimmunoassay method, an immunocolloidal gold technique, or Point of Care Testing (POCT);
and optionally wherein the immunoassay is ELISA.

20. The kit according to claim 19, wherein the sample is a blood, a tissue, a cell, a body fluid, an urine, or a fecal extract, and preferably, the sample is a blood.

21-24. (canceled)

25. A method for detecting a disease or a non-disease symptom, wherein the method comprises the following steps:
obtaining a sample from a subject;
using the claim 13 to detect the expression level of the dimer of S100A8 and S100A9 in the sample; and
determining that the subject has the disease or the non-disease symptom according to the expression level of the dimer of S100A8 and S100A9 in the sample.

26. The method according to claim 25, wherein the sample is a blood, a tissue, a cell, a body fluid, an urine, or a fecal extract, and preferably, the sample is a blood.

27. The method according to claim 25, wherein the disease or the non-disease symptom is associated with an increase in the expression level of the dimer of S100A8 and S100A9, and preferably, wherein the disease or the non-disease symptom is selected from sepsis, an autoimmune disease (such as systemic lupus erythematosus and ankylosing spondylitis), acute lung injury (such as chronic obstructive pulmonary disease and viral pneumonia), acute pancreatitis, acute kidney injury, tumors (such as pancreatic cancer), organ ischemia/reperfusion injury (such as myocardial ischemia/reperfusion injury, renal ischemia/reperfusion injury, liver ischemia/reperfusion injury and cerebral ischemia/reperfusion injury), and an atherosclerosis-associated cardio-cerebral disease (such as coronary heart disease, cerebral infarction and myocardial infarction).
